(19) United States
(12) Patent Application Publication
(10) Pub. No.: US 2005/0009109 A1
(43) Pub. Date: Jan. 13, 2005

(54) FLUOROPHORE COMPOUNDS AND THEIR USE IN BIOLOGICAL SYSTEMS

(75) Inventors: William E. MOERNER, Los Altos, CA (US); Robert J. TWIEG, Kent, OH (US); Douglas W. KLINE, Kent, OH (US); Meng HE, Monroeville, PA (US)

Correspondence Address: HOWREY SIMON ARNOLD & WHITE LLP, 750 BERING DRIVE, HOUSTON, TX, 77057, US

(73) Assignee: STANFORD UNIVERSITY, Palo Alto, CA (US)

(21) Appl. No.: 10/604,282

(22) Filed: Jul. 8, 2003

(51) Int. Cl. G01N 33/53

(52) U.S. Cl. 435/7.2

(57) ABSTRACT

Fluorophore compounds and methods for their use are disclosed. The fluorophores contain a 2-dicyanomethylen-3-cyano-2,5-dihydrofuran (DCDHF) moiety and one or more donor groups conjugated to the 2-dicyanomethylen-3-cyano-2,5-dihydrofuran group. The donor groups can contain atoms with free electron pairs such as oxygen, sulfur, nitrogen, or phosphorous. The fluorophore compounds can be used to label and detect biological molecules and biological structures either in vivo or in vitro.

### FEDERAL RESEARCH STATEMENT

The government may own rights in the present invention pursuant to grant number F49620-00-1-0038 from the U.S. Air Force Office of Scientific Research.

### BACKGROUND OF INVENTION

1. Field of the Invention

The invention relates to organic fluorophores and their use in labeling biomolecules and biological structures. In particular, organic molecules containing 2-dicyanomethylen-3-cyano-2,5-dihydrofuran (DCDHF) moieties and their use are disclosed.

2. Description of the Related Art

Many fluorescent compounds are widely used for visualizing targets of interest. The fluorescent compounds have traditionally been used for visualizing large numbers of targets. These compounds have typically been based on dyes such as rhodamines, cyanines, oxazines, or derivatives of rigid polynuclear aromatic hydrocarbons such as terrylene, perylene, and pyrene.

While these compounds have been effective in their various uses to date, the increasing interest in the study of molecules at the discrete or single molecule level is presenting new challenges and new demands for improved fluorescent compounds. Fluorophores for use in single molecule studies preferably show strong absorption, very high fluorescence quantum yield, weak bottlenecks into triplet states, and high photostability.

Gubler et al. described the preparation and use of 2-dicyanomethylen-3-cyano-5,5-dimethyl-4-(4″-dihexylaminophenyl)-2,5-dihydrofuran (DCDHF-6) in photorefractive organic glasses (Gubler, U. et al., Advanced Materials, 14(4): 313-317 (Feb. 19, 2002)). The compound was found to have very high photorefractive gain coefficients and speed in a PVK (polyvinylcarbazole) host matrix. The compound was also found to form an amorphous organic glass by itself.

He et al. described 2-dicyanomethylen-3-cyano-2,5-dihydrofuran derivative photorefractive materials, structure-property relationships, and their physical properties (He, M. et al., Proc. Soc. Photo-Opt. Instrum. Engr. 4802: 9-20 (2002)). A wide array of compounds was disclosed, and their thermal, UV-Vis, solvatochromic, and other properties were presented. A portion of this publication was presented on Jul. 9, 2002 at the International Symposium on Optical Science and Technology, SPIE 47^{th} Annual Meeting, Seattle, Wash., USA.

Willets et al. described six fluorophores useful for single-molecule imaging (Willets, K. A., et al., J. Am. Chem. Soc. Commun., 125: 1174-1175 (2003)). The molecules contained an amine donor and a 2-dicyanomethylen-3-cyano-2,5-dihydrofuran (DCDHF) acceptor linked by a conjugated unit (benzene, thiophene, alkene, styrene, 2-vinylthiophene). The properties of the fluorophores were studied at the single copy, individual molecule level as dopants in polymer films. All prior publications of DCDHF dyes were dominated by photorefractive applications and other electrooptic applications.

Fluorescent tags are commercially available from a wide array of suppliers such as Molecular Probes (Eugene, Oreg.), Biotium, Inc. (Hayward, Calif.), Panvera (Madison, Wis.), Vector Labs (Burlingame, Calif.), Sigma-Aldrich (St. Louis, Mo.), Biostatus (Leicestershire, UK), Atto-Tec (Siegen, Germany), Dyomics (Jena, Germany), Toronto Research Chemicals (North York, Ontario, Canada), and IBA (Goettingen, Germany).

While progress has been made steadily in the development of improved fluorophores, there still exists a need for enhanced fluorophores with demonstrated abilities to label biomolecules and biological structures. The ability to study labeled biomolecules and biological structures at the single molecule/structure level will be of great value to ongoing and future biological, chemical, and biomedical research.

### SUMMARY OF INVENTION

Fluorophore compounds containing at least one donor group conjugated to at least one 2-dicyanomethylen-3-cyano-2,5-dihydrofuran moiety are disclosed. Donor groups are commonly amines, but can be other atoms with lone pairs such as oxygen, sulfur and phosphorous. The fluorophore compounds can be used in methods to label, detect, and quantify biomolecules and biological structures. The fluorophore compounds can interact with the biomolecules and biological structures in a variety of manners such as by forming a covalent bond, by forming an ionic bond, by forming a pi-pi stacking interaction, by forming a hydrophobic interaction, or by van der Waals interactions.

### BRIEF DESCRIPTION OF DRAWINGS

The following figures form part of the present specification and are included to further demonstrate certain aspects of the present invention. The invention may be better understood by reference to one or more of these figures in combination with the detailed description of specific embodiments presented herein. Full chemical names were obtained using the Chemdraw Ultra software package, version 7.0.1.

FIG. 1 shows four fluorophore compounds. Structure 1 is DCDHF-MOE; 2-(4-{4-[Bis-(2-methoxy-ethyl)-amino]-phenyl}-3-cyano-5,5-dimethyl-5H-furan-2-ylidene)-malononitrile; structure 2 is DCDHF-1;
2-[3-Cyano-4-(4-dimethylamino-phenyl)-5,5-dimethyl-5H-furan-2-ylidene]-malononitrile; structure 3 is DCDHF-C6M;
2-[4-(4-Azepan-1-yl-phenyl)-3-cyano-5,5-dimethyl-5H-furan-2-ylidene]-malononitrile; and structure 4 is DCDHF-C5MDM;
2-{3-Cyano-4-[4-(3,5-dimethyl-piperidin-1-yl)-phenyl]-5,5-dimethyl-5H-furan-2-ylidene}-malononitrile.

FIG. 2 shows four fluorophore compounds. Structure 5 is DCDHF-2;
2-[3-Cyano-4-(4-diethylamino-phenyl)-5,5-dimethyl-5H-furan-2-ylidene]-malononitrile; structure 6 is DCDHF-3;
2-[3-Cyano-4-(4-dipropylamino-phenyl)-5,5-dimethyl-5H-furan-2-ylidene]-malononitrile; structure 7 is DCDHF-4;
2-[3-Cyano-4-(4-dibutylamino-phenyl)-5,5-dimethyl-5H-furan-2-ylidene]-malononitrile; and structure 8 is DCDHF-5;
2-[3-Cyano-4-(4-dipentylamino-phenyl)-5,5-dimethyl-5H-furan-2-ylidene]-malononitrile.

FIG. 3 shows three fluorophore compounds. Structure 9 is DCDHF-6;
2-[3-Cyano-4-(4-dihexylamino-phenyl)-5,5-dimethyl-5H-furan-2-ylidene]-malononitrile; structure 10 is DCDHF-8;
2-[3-Cyano-4-(4-dioctylamino-phenyl)-5,5-dimethyl-5H-furan-2-ylidene]-malononitrile; and structure 11 is DCDHF-2EH;
2-(4-{4-[Bis-(2-ethyl-hexyl)-amino]-phenyl}-3-cyano-5,5-dimethyl-5H-furan-2-ylidene)-malononitrile.

FIG. 4 shows three fluorophore compounds. Structure 12 is DCDHF-6-C7M;
2-[3-Cyano-4-(4-dihexylamino-phenyl)-1-oxa-spiro[4.7]dodec-3-en-2-ylidene]-malononitrile; structure 13 is DCDHF-6-DB;
2-[5,5-Dibutyl-3-cyano-4-(4-dihexylamino-phenyl)-5H-furan-2-ylidene]-malononitrile; and structure 14 is DCDHF-C6M-CF3;
2-[4-(4-Azepan-1-yl-phenyl)-3-cyano-5-methyl-5-trifluoromethyl-5H-furan-2-ylidene]-malononitrile.

FIG. 5 shows four fluorophore compounds. Structure 15 is DCDHF-6-CF3;
2-[3-Cyano-4-(4-dihexylamino-phenyl)-5-methyl-5-trifluoromethyl-5H-furan-2-ylidene]-malononitrile; structure 16 is DCDHF-2-CF3;
2-[3-Cyano-4-(4-diethylamino-phenyl)-5-methyl-5-trifluoromethyl-5H-furan-2-ylidene]-malononitrile; structure 17 is TH-DCDHF-6;
2-[3-Cyano-4-(5-dihexylamino-thiophen-2-yl)-5, 5-dimethyl-5H-furan-2-ylidene]-malononitrile; and structure 18 is TH-DCDHF-C6M;
2-[4-(5-Azepan-1-yl-thiophen-2-yl)-3-cyano-5,5-dimethyl-5H-furan-2-ylidene]-malononitrile.

FIG. 6 shows three fluorophore compounds. Structure 19 is TH-DCDHF-6-V;
2-{3-Cyano-4-[2-(5-dihexylamino-thiophen-2-yl)-vinyl]-5,5-dimethyl-5H-furan-2-ylidene}-malononitrile; structure 20 is DCDHF-2-V;
2-{3-Cyano-4-[2-(4-diethylamino-phenyl)-vinyl]-5,5-dimethyl-5H-furan-2-ylidene}-malononitrile; and structure 21 is DCDHF-J-V;
2-{3-Cyano-5,5-dimethyl-4-[2-(2,3,6,7-tetrahydro-1H,5H-pyrido[3,2,1-ij]quinolin-9-yl)-vinyl]-5H-furan-2-ylidene}-malononitrile.

FIG. 7 shows three fluorophore compounds. Structure 22 is DCDHF-6-V;
2-{3-Cyano-4-[2-(4-dihexylamino-phenyl)-vinyl]-5,5-dimethyl-5H-furan-2-ylidene}-malononitrile; structure 23 is DCDHF-2EH-V;
2-[4-(2-{4-[Bis-(2-ethyl-hexyl)-amino]-phenyl}-vinyl)-3-cyano-5,5-dimethyl-5H-furan-2-ylidene]-malononitrile; and structure 24 is DCDHF-MOE-V;
2-[4-(2-{4-[Bis-(2-methoxy-ethyl)-amino]-phenyl}-vinyl)-3-cyano-5,5-dimethyl-5H-furan-2-ylidene]-malononitrile.

FIG. 8 shows two fluorophore compounds. Structure 25 is DCDHF-DPH-V;
2-{3-Cyano-4-[2-(4-diphenylamino-phenyl)-vinyl]-5,5-dimethyl-5H-furan-2-ylidene}-malononitrile; and structure 26 is DCTA-6C-DCDHF-V;
2-[4-(2-{4-[(6-{4-[Bis-(4-carbazol-9-yl-phenyl)-amino]-phenoxy}-hexyl)-ethyl-amino]-phenyl}-vinyl)-3-cyano-5,5-dimethyl-5H-furan-2-ylidene]-malononitrile.

FIG. 9 shows three fluorophore compounds. Structure 27 is PFP-DDCDHF;
2-{3-Cyano-5,5-dimethyl-4-[1-(4-tridecafluorohexyl-phenyl)-1H-pyridin-4-ylidenemethyl]-5H-furan-2-ylidene}-malononitrile; structure 28 is HP-DDCDHF;
2-{3-Cyano-4-[1-(4-hexyl-phenyl)-1H-pyridin-4-ylidenemethyl]-5,5-dimethyl-5H-furan-2-ylidene}-malononitrile; and structure 29 is DOCP-DDCDHF;
4-[4-(4-Cyano-5-dicyanomethylene-2,2-dimethyl-2,5-dihydro-furan-3-ylmethylene)-4H-pyridin-1-yl]-benzoic acid dodecyl ester.

FIG. 10 shows three fluorophore compounds. Structure 30 is P-DDCDHF;
2-[3-Cyano-4-(2,6-dimethyl-1-phenyl-1H-pyridin-4-ylidenemethyl)-5,5-dimethyl-5H-furan-2-ylidene]-malononitrile; structure 31 is 2EHO-DDCDHF;
2-(3-Cyano-4-{1-[4-(2-ethyl-hexyloxy)-phenyl]-2,6-dimethyl-1H-pyridin-4-ylidenemethyl}-5,5-dimethyl-5H-furan-2-ylidene)-malononitrile; and structure 32 is M2EHO-DDCDHF;
2-(3-Cyano-4-{1-[3-(2-ethyl-hexyloxy)-phenyl]-2,6-dimethyl-1H-pyridin-4-ylidenemethyl}-5,5-dimethyl-5H-furan-2-ylidene)-malononitrile.

FIG. 11 shows one fluorophore compound. Structure 33 is DCDHF-2-2V;
2-{3-Cyano-4-[4-(4-diethylamino-phenyl)-buta-1,3-dienyl]-5,5-dimethyl-5H-furan-2-ylidene}-malononitrile.

FIG. 12 shows commercially available calcium tag R-1244 (structure 34), and a DCDHF structure covalently attached to a calcium Ca²⁺ ligand (structure 35).

FIG. 13 shows synthetic Scheme 1.

FIG. 14 shows synthetic Scheme 2.

FIG. 15 shows synthetic Scheme 3.

FIG. 16 shows synthetic Scheme 4.

FIG. 17 shows synthetic Scheme 5.

FIG. 18 shows synthetic Scheme 6.

FIG. 19 shows synthetic Scheme 7.

FIG. 20 shows synthetic Scheme 8.

FIG. 21 shows synthetic Scheme 9.

FIG. 22 shows designed example fluorophore compounds containing functional groups for interaction with biomolecules and biological structures. Structure 36 contains a maleimide reactive group
(2-(3-Cyano-4-{4-dimethylamino-3-[5-(2,5-dioxo-2,5-dihydro-pyrrol-1-yl)-pentyloxy]-phenyl}-5,5-dimethyl-5H-furan-2-ylidene)-malononitrile); structure 37 contains a methanethiosulfonate reactive group (Methanethiosulfonic acid S-[4-cyano-5-dicyanomethylene-3-(4-diethylamino-phenyl)-2-methyl-2,5-dihydro-furan-2-ylmethyl]ester).

### DETAILED DESCRIPTION

Organic fluorophore compounds are disclosed that are attractive for use in imaging biomolecules and biological structures. The compounds generically contain at least one 2-dicyanomethylen-3-cyano-2,5-dihydrofuran (“DCDHF”) moiety and one or more amine groups.

Fluorophore Compounds

Fluorophore compounds containing a 2-dicyanomethylen-3-cyano-2,5-dihydrofuran (DCDHF) moiety and one or more donor groups are disclosed. The general chemical structure for the fluorophore compounds is as follows (Structures I or II): 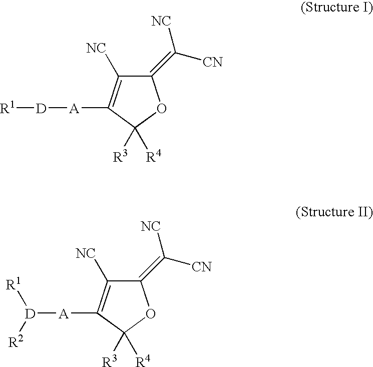
wherein: D is a donor group having at least one free electron pair conjugated with A, and A is a moiety having at least one multiple bond conjugated with the donor group and the 2-dicyanomethylen-3-cyano-2,5-dihydrofuran group. D and A can exist in the same ring structure in addition to being conjugated with each other. The choice between Structures I and II depends on the type of donor atom having at least one free electron pair conjugated with A. For example, if the donor atom is oxygen or sulfur, or nitrogen that shares a ring structure with A, then only one R group (R¹) is required to establish its proper valency, while if the atom is nitrogen (not sharing any ring structure with A) or phosphorous, then two R groups (R¹ and R²) are required to establish its proper valency. R¹ is an alkyl group, alkoxy alkyl group, aromatic group, substituted aromatic group, or hydrogen; R² is an alkyl group, alkoxy alkyl group, aromatic group, substituted aromatic group, or hydrogen; R³ is an alkyl group, fluoroalkyl group, aromatic group, or substituted aromatic group; and R⁴ is an alkyl group, fluoroalkyl group, aromatic group, or substituted aromatic group. R¹ and R² can be the same or different. R¹ and R² can be separate or can be joined to make a heteroatom-containing ring. If the donor group atom having at least one free electron pair is a nitrogen or phosphorous, the groups attached to it can be separate, or can form a ring containing the donor group atom. R³ and R⁴ can be the same or different. R³ and R⁴ can be separate or can be joined to make a ring.

Alkyl groups can include methyl, ethyl, propyl, butyl, pentyl, hexyl, heptyl, and octyl. Alkyl groups can include longer straight chain groups such as C₁₀H₂₁, C₁₂H₂₅, C₁₄H₂₉,C₁₆H₃₃, C₁₈H₃₇, C₂₀H₄₁, and C₂₂H₄₅. Alkyl groups can be straight chain, branched, or cyclic. Alkoxy groups can include methoxy and ethoxy. Alkoxy alkyl groups can include methoxymethyl, methoxyethyl, ethoxymethyl, and ethoxyethyl. Alkyl groups can also include substituted alkyl groups such as fluoroalkyl groups (e.g. trifluoromethyl or pentafluoroethyl), and containing other functionality (ketone, ester, aldehyde, carboxylic acid, amide, alcohol, nitrile, alkene, alkyne, and so on).

The A group can contain an aromatic group. For example, the A group can be a benzene ring or another aromatic system. The 2-dicyanomethylen-3-cyano-2,5-dihydrofuran group and the donor group atom (e.g. nitrogen, oxygen, or sulfur) can be in a 1,2 (ortho), 1,3 (meta), or 1,4 (para) arrangement across the benzene ring. The para arrangement is presently preferred. The A group can be a condensed aromatic system such as naphthalene, anthracene, phenanthrene, pyrene, and so on. The A group can also contain a carbon-carbon double bond (i.e. a vinyl group). For example, A can be a benzene ring linked to a double bond (styrene; C₆H₄—CH═CH—). The A group can also contain a carbon-carbon triple bond. For example, A can be a tolane (phenyl-C≡C-phenyl) group. The A group can include atoms other than carbon and hydrogen. For example, the A group can include oxygen, nitrogen, or sulfur. Examples of heterocycles with one heteroatom include thiophene, furan, and pyrrole, and examples of heterocycles with multiple heteroatoms include imidazole, pyrazole, oxazole, thiazole, diazole, oxadiazole, and thiadiazole. The heteroatom containing group can be condensed with benzene as in benzimidazole, benzoxazole, benzthiazole or contain multiple fused heterocycle rings such as thieno[3,2-b]thiophene and dithieno[3,2-b:2″,3″-d]thiophene. The A group can also have no ring and be comprised of one or more alkenes —(CH═CH)ₙ— and also imines (CH═N) and the two in conjunction.

A variety of example inventive fluorophore compounds are shown in the Figures. The fluorophore compound in compositions, but not for methods, is preferably not DCDHF-6 (2-[3-Cyano-4-(4-dihexylamino-phenyl)-5,5-dimethyl-5H-furan-2-ylidene]-malononitrile; where A is a benzene ring, D is dihexylamine, and both R³ and R⁴ are methyl).

Specific inventive fluorophore compounds include DCDHF-MOE
(2-(4-{4-[Bis-(2-methoxy-ethyl)-amino]-phenyl}-3-cyano-5,5-dimethyl-5H-furan-2-ylidene)-malononitrile), DCDHF-1
(2-[3-Cyano-4-(4-dimethylamino-phenyl)-5,5-dimethyl-5H-furan-2-ylidene]-malononitrile), DCDHF-C6M
(2-[4-(4-Azepan-1-yl-phenyl)-3-cyano-5,5-dimethyl-5H-furan-2-ylidene]-malononitrile), DCDHF-C5MDM
(2-{3-Cyano-4-[4-(3,5-dimethyl-piperidin-1-yl)-phenyl]-5,5-dimethyl-5H-furan-2-ylidene}-malononitrile), DCDHF-2
(2-[3-Cyano-4-(4-diethylamino-phenyl)-5,5-dimethyl-5H-furan-2-ylidene]-malononitrile), DCDHF-3
(2-[3-Cyano-4-(4-dipropylamino-phenyl)-5,5-dimethyl-5H-furan-2-ylidene]-malononitrile), DCDHF-4
(2-[3-Cyano-4-(4-dibutylamino-phenyl)-5,5-dimethyl-5H-furan-2-ylidene]-malononitrile), DCDHF-5
(2-[3-Cyano-4-(4-dipentylamino-phenyl)-5,5-dimethyl-5H-furan-2-ylidene]-malononitrile), DCDHF-8
(2-[3-Cyano-4-(4-dioctylamino-phenyl)-5,5-dimethyl-5H-furan-2-ylidene]-malononitrile), DCDHF-2EH
(2-(4-{4-[Bis-(2-ethyl-hexyl)-amino]-phenyl}-3-cyano-5,5-dimethyl-5H-furan-2-ylidene)-malononitrile), DCDHF-6-C7M
(2-[3-Cyano-4-(4-dihexylamino-phenyl)-1-oxa-spiro[4.7]dodec-3-en-2-ylidene]-malononitrile), DCDHF-6-DB
(2-[5,5-Dibutyl-3-cyano-4-(4-dihexylamino-phenyl)-5H-furan-2-ylidene]-malononitrile), DCDHF-C6M-CF3
(2-[4-(4-Azepan-1-yl-phenyl)-3-cyano-5-methyl-5-trifluoromethyl-5H-furan-2-ylidene]-malononitrile), DCDHF-6-CF3
(2-[3-Cyano-4-(4-dihexylamino-phenyl)-5-methyl-5-trifluoromethyl-5H-furan-2-ylidene]-malononitrile), DCDHF-2-CF3
(2-[3-Cyano-4-(4-diethylamino-phenyl)-5-methyl-5-trifluoromethyl-5H-furan-2-ylidene]-malononitrile), TH-DCDHF-6
(2-[3-Cyano-4-(5-dihexylamino-thiophen-2-yl)-5,5-dimethyl-5H-furan-2-ylidene]-malononitrile), TH-DCDHF-C6M
(2-[4-(5-Azepan-1-yl-thiophen-2-yl)-3-cyano-5,5-dimethyl-5H-furan-2-ylidene]-malononitrile), TH-DCDHF-6-V(2-{3-Cyano-4-[2-(5-dihexylamino-thiophen-2-yl)-vinyl]-5,5-dimethyl-5H-furan-2-ylidene}-malononitrile), DCDHF-2-V
(2-{3-Cyano-4-[2-(4-diethylamino-phenyl)-vinyl]-5,5-dimethyl-5H-furan-2-ylidene}-malononitrile), DCDHF-J-V
(2-{3-Cyano-5,5-dimethyl-4-[2-(2,3,6,7-tetrahydro-1H, 5H-pyrido[3,2, 1-ij]quinolin-9-yl)-vinyl]-5H-furan-2-ylidene}-malononitrile), DCDHF-6-V
(2-{3-Cyano-4-[2-(4-dihexylamino-phenyl)-vinyl]-5,5-dimethyl-5H-furan-2-ylidene}-malononitrile), DCDHF-2EH-V
(2-[4-(2-{4-[Bis-(2-ethyl-hexyl)-amino]-phenyl}-vinyl)-3-cyano-5,5-dimethyl-5H-furan-2-ylidene]-malononitrile), DCDHF-MOE-V
(2-[4-(2-{4-[Bis-(2-methoxy-ethyl)-amino]-phenyl}-vinyl)-3-cyano-5,5-dimethyl-5H-furan-2-ylidene]-malononitrile), DCDHF-DPH-V
(2-{3-Cyano-4-[2-(4-diphenylamino-phenyl)-vinyl]-5,5-dimethyl-5H-furan-2-ylidene}-malononitrile), DCTA-6C-DCDHF-V
(2-[4-(2-{4-[(6-{4-[Bis-(4-carbazol-9-yl-phenyl)-amino]-phenoxy}-hexyl)-ethyl-amino]-phenyl}-vinyl)-3-cyano-5,5-dimethyl-5H-furan-2-ylidene]-malononitrile), PFP-DDCDHF
(2-{3-Cyano-5,5-dimethyl-4-[1-(4-tridecafluorohexyl-phenyl)-1H-pyridin-4-ylidenemethyl]-5H-furan-2-ylidene}-malononitrile), HP-DDCDHF
(2-{3-Cyano-4-[1-(4-hexyl-phenyl)-1H-pyridin-4-ylidenemethyl]-5,5-dimethyl-5H-furan-2-ylidene}-malononitrile), DOCP-DDCDHF
(4-[4-(4-Cyano-5-dicyanomethylene-2,2-dimethyl-2,5-dihydro-furan-3-ylmethylene)-4H-pyridin-1-yl]-benzoic acid dodecyl ester), P-DDCDHF
(2-[3-Cyano-4-(2,6-dimethyl-1-phenyl-1H-pyridin-4-ylidenemethyl)-5,5-dimethyl-5H-furan-2-ylidene]-malononitrile), 2EHO-DDCDHF
(2-(3-Cyano-4-{1-[4-(2-ethyl-hexyloxy)-phenyl]-2,6-dimethyl-1H-pyridin-4-ylidenemethyl}-5,5-dimethyl-5H-furan-2-ylidene)-malononitrile), M2EHO-DDCDHF
(2-(3-Cyano-4-{1-[3-(2-ethyl-hexyloxy)-phenyl]-2,6-dimethyl-1H-pyridin-4-ylidenemethyl}-5,5-dimethyl-5H-furan-2-ylidene)-malononitrile), and DCDHF-2-2V
(2-{3-Cyano-4-[4-(4-diethylamino-phenyl)-buta-1,3-dienyl]-5,5-dimethyl-5H-furan-2-ylidene}-malononitrile).

The fluorophore compound can be cationic, anionic, neutral, or zwitterionic in charge. The fluorophore compound can be hydrophobic, hydrophilic, or amphiphilic. The fluorophore compounds may feature large ground-state electric dipole moments and a large polarizability anisotropy. The compounds may reorient as a result of changes in biological or other environmental and applied electric fields. Due to the large polarizability anisotropy, the compounds may lead to optical signals that can be detected with the optical polarization. The compounds may have large molecular hyperpolarizabilities. Due to the large hyperpolarizability, the compounds may generate light of twice the incident energy via a nonlinear optical interaction (second harmonic generation). The fluorescence emission efficiency of the compounds may depend strongly upon the ability of the functional groups of the molecule to reorient during the optical interaction. For example, the groups R¹ and R² may rotate in a nonrestrictive environment leading to reduced emission by a twisted intermolecular charge transfer state. On the other hand, in a constrained environment, these groups may not rotate, and the emission may be increased. For example, in a nonrestrictive environment the molecule might isomerize again leading to reduced emission, while in a restricted environment, isomerization may be reduced and the emission may increase. The compounds can cover a wide wavelength range, from green to far red. An example of such a range is about 400 nm to about 1200 nm. This allows the compounds to be used in multicolor labeling and/or fluorescence resonant energy transfer (FRET). The compounds may be responsive to viscosity, temperature, pressure, pH, and other environmental factors. The compounds may also exhibit multi-photon interactions and two-photon fluorescence.

The fluorophore compound can further comprise at least one functional group suitable for formation of a covalent bond with a biomolecule or biological structure. This functional group can include a thiol group (—SH), a maleimide group (for attachment to thiols), an iodoacetamide group (for attachment to thiols), an N-hydroxy-succinimide group (for attachment to amines), a phosphoramidite group, and a methanethiosulfonate group. The functional group can be located in a variety of locations within the fluorophore compound. For example, the functional group can be located at D, R¹, R², R³, R⁴, or A.

The functional group can be directly covalently attached to the fluorophore compound, or can be connected via a linker group. The linker group can be short or long. Short linker groups may be desirable to minimize internal twisting, and to facilitate interaction of the fluorophore compound with a biomolecule or biological structure. Long linker groups may be desirable to facilitate fast rotation or to minimize steric interferences. Linker groups can generally be any of the alkyl groups, alkoxy alkyl groups, aromatic groups, or substituted aromatic groups described above. Straight chain alkyl or alkoxy alkyl groups are commonly used as linker groups.

Methods of Use

Any of the above described fluorophore compounds can be used for labeling and visualizing biomolecules and biological structures. The methods of use can involve in vitro applications or in vivo applications.

Biomolecules that can be labeled include DNA, RNA, monosaccharides, polysaccharides, nucleotides (ATP, GTP, cAMP), lipids, peptides, and proteins (including enzymes and other structural proteins). Biological structures such as lipid bilayers, membranes, micelles, transmembrane proteins, ribosomes, liposomes, nucleosomes, peroxisomes, cytoskeletal units, plastids, chloroplasts, or mitochondria, can also be labeled using the fluorophore compounds. The biomolecules and biological structures can interact with the fluorophore compounds in a variety of manners. For example, the interaction can be through a covalent bond, through an ionic bond, through a pi-pi stacking interaction, through hydrophobic interactions, through ampiphilic interactions, through van der Waals interactions, fluorophore-fluorophore interactions, and so on. The interaction can be reversible or irreversible. The interaction can be with the surface of the biomolecules and biological structures, or the fluorophore compound can interact with an interior cavity, binding site, or other available structure or space.

Fluorophore compounds can be designed and selected for their ability to form covalent bonds with various biological molecules. For example, fluorophore compounds containing maleimide, acetamide, or methanethiosulfonate groups can covalently react with thiol groups such as found in protein or peptide cysteine residues. N-hydroxy-succinimide groups can be used to covalently attach to amine groups such as found in protein or peptide lysine groups. Phosphoramidite groups can be used to covalently attach the fluorophore compounds to nucleic acids such as DNA or RNA.

Labeling methods can involve contacting the biomolecules with at least one fluorophore compound under conditions suitable for labeling. Typically, the labeling will be performed in a liquid solution with other chemical agents present. The additional chemical agents can include salts, buffers, detergents, and so on. The liquid solution can also include water and/or other solvents such as methanol, ethanol, dimethylsulfoxide (DMSO), and tetrahydrofuran (THF).

The in vivo applications can involve contacting the fluorophore compound with cells suspended in culture, with cells immobilized on a surface, with a slice of tissue, with a monolayer of cells, with a tissue, or with an intact organism. For example, the fluorophore compounds may directly insert into the membrane of the cell. The in vivo applications can further comprise a step of enhancing the ability of the target cells to uptake the fluorophore compound. The enhancing step can comprise treating the cells with a detergent, treating the cells with dimethylsulfoxide (DMSO), treating the cells with one or more pulses of an electrical charge (electroporation), or treating the cells briefly with osmotic shock. Alternatively, the contacting step can comprise direct injection of the fluorophore compound into the cell using a micropipette or other syringe devices.

The liquid solution can generally be at any pH compatible with the biomolecule and the fluorophore compound. For example, the pH can be about 5, about 5.5, about 6, about 6.5, about 7, about 7.5, about 8, about 8.5, about 9, and ranges between any two of these values.

The liquid solution can generally be at any temperature compatible with the biomolecule and the fluorophore compound. Typically, the liquid solution will be at a temperature of about 0° C. to about 50° C. Temperatures can be about 0° C., about 5° C., about 10° C., about 15° C., about 20° C., about 25° C., about 30° C., about 35° C., about 40° C., about 45° C., about 50° C., and ranges between any two of these values.

The contacting step can generally be performed for any suitable length of time. For example, the contacting step can be performed for about 1 minute, about 2 minutes, about 3 minutes, about 4 minutes, about 5 minutes, about 10 minutes, about 20 minutes, about 30 minutes, about 40 minutes, about 50 minutes, about 1 hour, about 2 hours, about 3 hours, about 4 hours, about 5 hours, or ranges between any two of these values.

The methods of use can further comprise a purification step performed after the contacting step. The purification step can comprise separating unbound fluorophore compound from fluorophore compound bound to the biomolecules. The purification step can comprise the use of chromatography (such as agarose gel electrophoresis, polyacrylamide gel electrophoresis (“PAGE”), SDS-polyacrylamide gel electrophoresis (“SDS-PAGE”), isoelectric focusing, affinity chromatography, separation with magnetic particles, ELISA, HPLC, FPLC, centrifugation, density gradient centrifugation, dialysis, or osmosis.

The methods of use can further comprise visualizing the fluorophore compound bound to the biomolecules. The visualization can be performed by illumination by a light source followed by epifluorescence microscopy, by total internal reflection fluorescence microscopy, by confocal microscopy, by two-photon or three-photon emission microscopy, by second harmonic imaging microscopy, by polarization microscopy, or by aperture-based or apertureless near-field optical microscopy. The methods of use can further comprise quantifying the fluorophore compound bound to the biomolecules. The quantification can be performed by counting detected photons in a time interval, by pumping the fluorophore with light of different polarizations, by measuring the polarization of the detected photons, by measuring the anisotropy of the detected photons, by measuring the spectrum of the detected photons, by measuring the lifetime of the detected photons, or by measuring the correlations of the detected photons. Correlations can be measured by fluorescence correlation spectroscopy, by start-stop coincidence counting, by using hardware autocorrelators, or by time-tagging the emission time of each photon with respect to the time of a pumping light pulse followed by off-line computation.

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the scope of the invention.

### EXAMPLES

### Example 1

### Preparation of 2-methyl-2-trimethylsilyloxypropionitrile 5

A mixture of acetone cyanohydrin (160 ml, 1.26 mol) and pyridine (90 ml, 1.24 mol) was stirred in an ice bath under the protection of dry nitrogen. Neat TMSCI (100 ml, 1.1 mol) was then slowly added via a dropping funnel at 0° C. After the addition, a large quantity of a white solid was produced and the reaction mixture was kept stirring at room temperature for 8 hours more. The reaction mixture was slowly poured into a vigorously stirred mixture of 200 ml saturated sodium bicarbonate solution and 200 ml petroleum ether. After stirring for one hour the organic layer was isolated in a separatory funnel, washed several times with water and dried over magnesium sulfate. After filtration, the petroleum ether was removed by distillation and the residue was purified by vacuum distillation at 75-100 kPa. Material boiling at 110° C. was collected to give 160 g (92% yield) of clear liquid: ¹H NMR (300 MHz, CDCl₃) δ 0.20 (s, 9 H), 1.57 (s, 6 H); ¹³C NMR (75 MHz, CDCl₃) δ 1.38, 30.98, 66.25, 122.82 ppm.

### Example 2

### Preparation of 1-(4-fluorophenyl)-2-hydroxy-2-methylpropan-1-one 7

Under the protection of nitrogen, a solution of 4-bromofluorobenzene (59 g, 0.34 mol) in dry THF (60 ml) was added dropwise at room temperature to a stirred mixture of magnesium turnings (9.84 g, 0.405 mol) in 20 ml of dry THF containing four drops of 1,2-dibromoethane. An ice water bath was occasionally used to moderate the reaction temperature. The addition was finished in two hours and stirring was maintained for one more hour at room temperature. A solution of 5 (53 g, 0.34 mol) in 60 ml dry THF was added dropwise to the solution of the Grignard reagent and the mixture was stirred at room temperature for 16 hours. After this time large quantities of white precipitate could be observed and 340 ml 6 N HCl was carefully added into the mixture with ice cooling and vigorous stirring. The mixture was then stirred at room temperature for 4 more hours until TLC showed only one major spot and then sodium bicarbonate was used to neutralize the excess acid and the solid in the mixture was removed by vacuum filtration through a pad of Celite. The filtrate was extracted with ethyl acetate, dried over anhydrous MgSO₄ and after evaporation of the solvent 83 g of liquid was obtained which was suitable for direct use in the next step was obtained. For further characterization, one gram of this liquid was purified by column chromatography (solvent: EtOAc/hexane=1/4) to give 0.65 g clear liquid (calculated yield 88%), which solidified upon standing in vacuum at room temperature as colorless crystals: mp 131° C. ¹H NMR (300 MHz, CDCl₃) δ 1.54 (s, 6 H), 4.13 (s, 1 H), 7.06 (dd, J=9.0, 8.7 Hz, 2 H), 8.07 (dd, J=9.0, 5.4 Hz, 2 H); ¹³C NMR (75 MHz, CDCl₃) δ 28.31, 76.73, 115.5 (d, J=21.6 Hz), 128.7 (d, J=7.9 Hz), 132.7, 166 (d, J=243 Hz), 202.87 ppm; ¹⁹F NMR (282 MHz, CDCl₃) δ 105.00 (tt, J=5.4, 8.7 Hz, 1 F).

### Example 3

### Preparation of 1-(4-dihexylaminophenyl)-2-hydroxy-2-methyl-propan-1-one (8a with R₁=R₂=hexyl)

A two steps procedure was used to synthesize the title compound.

The synthesis of 4-bromo-N,N-(dihexyl)aniline 3a: A mixture of 4-bromoaniline (15 g, 87.2 mmol), n-hexylbromide (43.2 g, 262 mmol) and potassium hydroxide (14.65 g, 262 mmol) was stirred at 150° C. for 8 hours. After the reaction was complete 200 ml water was added and the mixture was extracted with ethyl acetate. The organic layer was then washed with water, dried over magnesium sulfate and concentrated in vacuum. The obtained crude product was purified by Kugelrohr distillation to give 27 g (yield 91%) of clear liquid, which is 4-bromo-N,N-(dihexyl)aniline: ¹H NMR (300 MHz, CDCl₃) δ 0.92 (t, J=6.6 Hz, 6 H), 1.32 (m, 12 H), 1.56 (m, 4 H), 3.23 (t, J=7.5 Hz, 4 H), 6.50 (d, J=9.0 Hz, 2 H), 7.26 (d, J=9.0 Hz, 2 H); ¹³C NMR (75 MHz, CDCl₃) δ 14.12, 22.76, 26.88, 27.11, 31.80, 51.21, 106.77, 113.37, 131.84, 147.18 ppm.

Under the protection of nitrogen, a solution of 4-bromo-N,N-(dihexyl)aniline3a (14.43 g, 42.4 mmol) in dry THF (20 ml) was added dropwise at room temperature to a stirred mixture of magnesium turnings (1.134 g, 46.6 mmol), 5 ml dry THF and two drops of 1,2-dibromoethane, after which stirring was maintained for two more hours at room temperature until GC showed no starting bromide. A solution of 5 (6.67 g, 42.4 mmol) in 10 ml dry toluene was then added to the Grignard mixture via a dropping funnel. The mixture was stirred at room temperature for 6 hours and then 38 ml 6 N HCl was added into the mixture carefully under ice cooling and vigorous stirring. The mixture was then stirred at room temperature for 4 more hours. Solid sodium bicarbonate was used to neutralize the excess acid. The solids in the mixture were filtered off over a pad of Celite by vacuum filtration. The liquid obtained was then extracted by EtOAc. After drying the organic solution over anhydrous MgSO₄ and evaporation of the solvent, the 16 g residue was purified by column chromatography (solvent: EtOAc/hexane=1/9) to give 11.3 g (77% yield) clear liquid: ¹H NMR (300 MHz, CDCl₃) δ 0.94 (t, J=6.3 Hz, 6 H), 1.33 (m, 12 H), 1.60 (m, 4 H), 1.64 (s, 6 H), 3.33 (t, J=7.8 Hz, 4 H), 4.82 (s, 1 H), 6.59 (d, J=9.3 Hz, 2 H), 7.96 (d, J=9.3 Hz, 2 H); ¹³C NMR (75 MHz, CDCl₃) δ 14.00, 22.65, 26.76, 27.23, 29.09, 31.67, 51.05, 74.98, 110.22, 119.15, 132.73, 151.68, 201.15 ppm.

### Example 4

### Preparation of 1-(4-dimethylaminophenyl)-2-hydroxy-2-methyl-propan-1-one (8b with R₁=R₂=methyl)

In the same way described already for 8a, 8b was obtained as light yellow crystals: mp 112.9° C. (lit. 115° C., Merck Patent; DE 2808459; 1978; Chem.Abstr.; EN; 92; 6245). ¹H NMR (300 MHz, CDCl₃) δ 1.63 (s, 6 H), 3.07 (s, 6 H), 4.69 (s, 1 H), 6.66 (d, J=9.3 Hz, 2 H), 7.99 (d, J=9.3 Hz, 2 H); ¹³C NMR (75 MHz, CDCl₃) δ 29.13, 40.01, 75.21, 110.67, 120.31, 132.56, 153.54, 201.71; IR (neat, cm⁻¹) 3439, 2982, 1643, 1586, 1371.

### Example 5

### Preparation of 3-cyano-2-dicyanomethylen-4-(4-fluorophenyl)-5,5-dimethyl-2,5-dihydrofuran 9

A mixture of the crude product 7(82 g, 65%, 0.29 mol), malononitrile (90 g, 1.36 mol), acetic acid (0.8 g) and pyridine (350 ml) was stirred at room temperature for 24 hours. The reaction mixture was then poured into 4 L of ice water with vigorous stirring and the resulting mixture was left standing in a refrigerator overnight. The green precipitate was collected by vacuum filtration and washed several times with methanol to give 48 g (59% yield) of a green solid which is of sufficient purity for direct use in the next step: mp 263° C. ¹H NMR (300 MHz, CDCl₃) δ 1.82 (s, 6 H), 7.31 (dd, J=9.0 Hz, 8.1 Hz, 2 H), 7.82 (dd, J=9.0 Hz, 4.8 Hz, 2 H); ¹⁹F NMR (282 MHz, CDCl₃) δ −101.78 (tt, J=4.8, 8.1 Hz, 1 F).

### Example 6

### Preparation of 3-cyano-2-dicyanomethylen-5,5-dimethyl-4-[4″-(N,N-dioctylamino)phenyl]-2,5-dihydrofuran (Entry 10, DCDHF-8)

A mixture of 9 (3 g, 10.7 mmol), di-n-octylamine (7.8 g, 32.3 mmol) and 30 ml pyridine was stirred at room temperature for 24 hours. The mixture was poured into 500 ml water and this mixture was kept standing in a refrigerator overnight. The red solid that precipitated was collected by vacuum filtration and recrystallized from CH₂Cl₂/MeOH to give 3.5 g (65% yield) of red crystals: mp 123° C. ¹H NMR (300 MHz, CDCl₃) δ 0.89 (t, J=7.2 Hz, 6 H), 1.28-1.58 (m, 24 H), 1.82 (s, 6 H), 3.39 (t, J=8.0 Hz, 4 H), 6.72 (d, J=9.3 Hz, 2 H), 7.98 (d, J=9.3 Hz, 2 H); ¹³C NMR (75 MHz, CDCl₃) δ 14.22, 22.73, 27.08, 27.49, 27.80, 29.34, 29.47, 31.87, 51.53, 53.41, 90.07, 97.43, 112.18, 112.38, 113.12, 113.32, 113.56, 132.75, 153.11, 173.79, 177.39.

### Example 7

### Preparation of 3-cyano-2-dicyanomethylen-4-{4″-[N,N-(dimethoxyethyl)aminophenyl]}-5,5-dimethyl-2,5-dihydrofuran (Entry 1, DCDHF-MOE)

In the same way described already for DCDHF-8, dihydrofuran 9 (1.5 g, 5.3 7 m mol) was reacted with di(2-methoxyethyl)amine (4.3 g, 10.75 mmol) in pyridine (20 ml) to give dye DCDHF-MOE as red crystals (1.09 g, 52%): mp 183° C. ¹H NMR (300 MHz, CDCl₃) δ 1.82 (s, 6 H), 3.34 (s, 6 H), 3.60 (t, J=5.1 Hz, 4 H), 3.72 (t, J=5.1 Hz, 4 H), 6.84 (d, J=9.0 Hz, 2 H), 7.98 (d, J=9.0 Hz, 2 H); ¹³C NMR (75 MHz, CDCl₃) δ 27.62, 51.30, 54.13, 59.17, 70.01, 91.30, 97.51, 112.00, 112.55, 112.94, 113.10, 113.89, 132.36, 153.55, 174.10, 177.07 ppm. IR (neat, cm⁻¹) 2988, 2930, 2881, 2222, 1610, 1566, 1542, 1492, 1468, 1448, 1417, 1397, 1371, 1352, 1332, 1275, 1237, 1218, 1187, 1112, 985, 958, 920, 832.

### Example 8

### Preparation of 4-[4″-(azepan-1-yl)phenyl]-3-cyano-2-dicyanomethylen-5,5-dimethyl-2,5-dihydrofuran (Entry 3, DCDHF-C6M)

In the same way described already for DCDHF-8, dihydrofuran 9(1 g, 3.58 mmol) was reacted with azepane (1.1 g, 11 mmol) in pyridine (10 ml) to give dye DCDHF-C6M as red crystals (0.77 g, 60%): mp 249° C. ¹H NMR (300 MHz, CDCl₃) δ 1.55-1.88 (m, 8 H), 1.83 (s, 6 H), 3.61 (d, J=6.2 Hz, 4 H), 6.77 (d, J=9.3 Hz, 2 H), 7.99 (d, J=9.3 Hz, 2 H); ¹³ C NMR (75 MHz, CDCl₃) δ 26.64, 27.17, 27.82, 50.20, 54.05, 90.80, 97.35, 112.00, 112.19, 113.20, 113.42, 113.46, 132.75, 153.67, 173.75, 177.24.

### Example 9

### Preparation of 3-cyano-2-dicyanomethylen-5,5-dimethyl-4-[4″-(3,5-dimethylpiperidin-1-yl)phenyl]-2,5-dihydrofuran (Entry 4, DCDHF-C5MDM)

In the same way described already for DCDHF-8, dihydrofuran 9 (1 g, 3.58 mmol) was reacted with 3,5-dimethylpiperidine (1 g, 8.8 mmol) in pyridine (10 ml) to give dye DCDHF-C5MDM as red crystals (0.84 g, 63%): mp 305° C.; ¹³C NMR (75 MHz, CDCl₃) δ 19.22, 27.72, 31.23, 42.35, 54.29, 54.53, 91.47, 97.54, 112.11, 112.79, 113.11, 113.32, 113.96, 132.69, 153.88, 173.81, 177.16.

### Example 10

### Preparation of 3-cyano-2-dicyanomethylen-4-[4″-(N,N-diethylaminophenyl)]-5,5-dimethyl-2,5-dihydrofuran 44 (Entry 5, DCDHF-2)

In the same way described already for DCDHF-8, dihydrofuran 9 (1 g, 3.58 mmol) was reacted with diethylamine (0.79 g, 10.80 mmol) in pyridine (10 ml) to give dye DCDHF-2 as red crystals (0.6 g, 50%): mp 250.2° C. ¹H NMR (300 MHz, CDCl₃) δ 1.27 (t, J=7.2 Hz, 6 H), 1.83 (s, 6 H), 3.50 (q, 4 H), 6.77 (d, J=9.3 Hz, 2 H), 7.99 (d, J=9.3 Hz, 2 H); ¹³C NMR (75 MHz, CDCl₃) δ 12.71, 27.82, 45.32, 53.91, 90.68, 97.41, 112.03, 112.25, 113.26, 113.30, 113.44, 132.78, 152.63, 173.87, 177.30 ppm.

### Example 11

### Preparation of 3-cyano-2-dicyanomethylen-5,5-dimethyl-4-[4″-(N,N-dipropylaminophenyl)]-2,5-dihydrofuran (Entry 6, DCDHF-3)

In the same way described already for DCDHF-8, dihydrofuran 9 (1 g, 3.6 mmol) was reacted with dipropylamine (1.1 g, 11 mmol) in pyridine (10 ml) to give dye DCDHF-3 as red crystals (0.7 g, 54%): mp 278° C.; ¹H NMR (300 MHz, CDCl₃) δ 0.99(t, J=7.4 Hz, 6 H), 1.70 (m, 4 H), 1.83 (s, 6 H), 3.39 (t, J=7.8 Hz, 4 H), 6.76 (d, J=9.3 Hz, 2 H), 7.98 (d, J=9.3 Hz, 2 H); ¹³C NMR (75 MHz, CDCl₃) δ 11.43, 20.72, 27.80, 53.14, 54.03, 90.73, 97.31, 112.13, 112.33, 113.20, 113.23, 113.44, 132.62, 153.03, 173.71, 177.23 ppm; IR (neat, cm⁻¹) 2224 (CN).

### Example 12

### Preparation of 3-cyano-2-dicyanomethylen-4-[4″-(N,N-dibutylaminophenyl)]-5,5-dimethyl-2, 5-dihydrofuran (Entry 7, DCDHF-4)

In the same way described already for DCDHF-8, dihydrofuran 9 (0.5 g, 1.8 mmol) was reacted with dibutylamine (1.39 g, 11 mmol) in pyridine (15 ml) to give dye DCDHF-4 as red crystals (0.49 g, 70%): mp 250° C. ¹H NMR (300 MHz, CDCl₃) δ 0.98 (t, J=7.2 Hz, 6 H), 1.40 (m, 4 H), 1.62 (m, 4 H), 1.82 (s, 6 H), 3.41 (t, J=7.7 Hz, 4 H), 6.71 (d, J=9.3 Hz, 2 H), 7.99 (d, J=9.3 Hz, 2 H); ¹³C NMR (75 MHz, CDCl₃) δ 14.04, 20.36, 27.83, 29.54, 51.32, 53.73, 90.41, 97.40, 112.20, 112.34, 113.22, 113.32, 113.54, 132.71, 153.02, 173.78, 177.35 ppm.

### Example 13

### Preparation of 3-cyano-2-dicyanomethylen-5,5-dimethyl-4-[4″-(N,N-dipentylaminophenyl)]-2,5-dihydrofuran (Entry 8, DCDHF-5)

In the same way described already for DCDHF-8, dihydrofuran 9 (1 g, 3.6 mmol) was reacted with dipentylamine (1.7 g, 11 mmol) in pyridine (10 ml) to give dye DCDHF-5 as red crystals (0.75 g, 50%): mp 169° C.; ¹H NMR (300 MHz, CDCl₃) δ 0.92 (t, J=6.8 Hz, 6 H), 1.35 (m, 8 H), 1.64 (m, 4 H), 1.82 (s, 6 H), 3.40 (t, J=7.8 Hz, 4 H), 6.72 (d, J=9.3 Hz, 2 H), 7.99 (d, J=9.3 Hz, 2 H); ¹³C NMR (75 MHz, CDCl₃) δ 14.19, 22.65, 27.17, 27.82, 29.23, 51.59, 53.81, 90.52, 97.40, 112.26, 112.31, 113.29, 113.33, 113.51, 132.71, 152.95, 173.76, 177.32 ppm.

### Example 14

### Preparation of 3-cyano-2-dicyanomethylen-4-[4″-(N,N-dihexylaminophenyl)]-5,5-dimethyl-2,5-dihydrofuran (Entry 9, DCDHF-6)

Preparation method A: in the same way described already for DCDHF-8, dihydrofuran 9 (1 g, 3.6 mmol) was reacted with dihexylamine (2.0 g, 11 mmol) in pyridine (10 ml) to give dye DCDHF-6 as red crystals (1.1 g, 68%) Preparation method B from 8a (R₁=R₂=hexyl): A mixture of 8a (R₁=R₂=hexyl) (4.93 g, 14.2 mmol), malononitrile (2.81 g, 42.5 mmol), acetic acid (0.08 g), ammonium acetate (0.02 g), 3 Å molecular sieves (5 g) and pyridine (30 ml) was stirred at room temperature for 24 hours. The reaction mixture was then poured into 300 ml ice water with vigorous stirring and the resulting mixture was left standing in a refrigerator overnight. The produced red precipitate was collected by vacuum filtration, dissolved in EtOAc and dried over MgSO₄. The solid was then filtrated off over a pad of Celite. After evaporation of the solvent, the oily residue was crystallized by the addition of hexane. The red crystals was collected and recrystallized from CH₂Cl₂/MeOH (3.71 g, yield 59%): mp 129° C. (127° C. from second heating in DSC); ¹H NMR (300 MHz, CDCl₃) δ 0.90 (t, J=6.6 Hz, 6 H), 1.34 (m, 12 H), 1.63 (m, 4 H), 1.82 (s, 6 H), 3.40 (t, J=7.8 Hz, 4 H), 6.70 (d, J=9.6 Hz, 2 H), 7.99 (d, J=9.6 Hz, 2 H); ¹³C NMR (75 MHz, CDCl₃) δ 13.97, 22.60, 26.66, 27.37, 27.69, 31.57, 51.43, 53.74, 90.43, 97.23, 112.06 (2 carbons), 113.08, 113.13, 113.32, 132.57, 152.98, 173.66, 177.15 ppm; IR (neat, cm⁻¹) 2950, 2928, 2856, 2223, 1607, 1564, 1538, 1491, 1472, 1422, 1355, 1333, 1220, 1187, 1119, 1002, 981, 920, 826; UV-Vis (THF) λₘₐₓnm (ε) 491 (72455 L mol⁻¹ cm⁻¹).

### Example 15

### Preparation of 3-cyano-2-dicyanomethylen-4-[4″-(N,N-dihexylaminophenyl)]-5,5-dimethyl-2,5-dihydrofuran (Entry 2, DCDHF-1)

As same as preparation method B described for DCDHF-6, DCDHF-1 was prepared from 8b as black crystals: mp>300° C. ¹H NMR (300 MHz, CDCl₃) δ 1.83 (s, 6 H), 3.18 (s, 6 H), 6.76 (d, J=9.6 Hz, 2 H), 8.00 (d, J=9.6 Hz, 2 H).

### Example 16

### Preparation of 3-cyano-2-dicyanomethylen-4-{4″-[N,N-di-(2-ethylhexyl)]aminophenyl}-5,5-dimethyl-2,5-dihydrofuran (Entry 11, DCDHF-2EH)

A mixture of 9 (5 g, 18 mmol), di-(2-ethylhexyl)amine (14 g, 58 mmol), pyridine (40 ml) and hexamethylphosphoramide (30 ml) was stirred at 60° C. for 48 hours. The mixture was poured into 500 ml water and this mixture was extracted with ethyl acetate. The crude product was purified by column chromatography (solvent: EtOAc/hexane=1/9) followed by recrystallization from CH₂Cl₂/methanol to give orange crystals (0.48 g, 5.4%): mp 171° C. ¹H NMR (300 MHz, CDCl₃) δ 0.82 (m, 12 H), 1.28 (m, 18 H), 1.83 (s, 6 H), 3.38 (m, 4 H), 6.72 (d, J=9.3 Hz, 2 H), 7.97 (d, J=9.3 Hz, 2 H); ¹³C NMR (75 MHz, CDCl₃) δ 10.77, 14.16, 23.24, 23.95, 27.83, 28.68, 30.64, 37.56, 53.84, 56.59, 90.55, 97.35, 112.24, 112.86, 113.22 (2 carbons), 113.46, 132.43, 153.29, 173.66, 177.26 ppm.

### Example 17

### Preparation of 1-trimethylsilyloxycyclooctylcarbonitrile 12

A mixture of TMSCN (15 ml, 112 mmol), cyclooctanone (12.84 g, 102 mmol) and dry THF (150 ml) was stirred in a flame-dried flask and chilled in an ice bath while protected under dry nitrogen. A catalytic amount of n-BuLi (2.5 M in hexanes, 0.1 ml) was added via syringe at 0° C. After stirring at room temperature for 4 hours, the mixture was Kugelrohr distilled. The product was collected as a clear liquid of 22.27 g (yield 99%). ¹H NMR (300 MHz, CDCl₃) δ 0.22 (s, 9 H), 1.60 (m, 10 H), 2.00 (t, J=6.0 Hz, 4 H); ¹³C NMR (75 MHz, CDCl₃) δ 1.38, 21.10, 24.15, 27.62, 37.28, 73.06, 122.85.); ¹³C NMR (75 MHz, CDCl₃) δ 1.38, 21.10, 24.15, 27.62, 37.28, 73.06, 122.85.

### Example 18

### Preparation of 1-{4″-[N,N-(dihexyl)aminobenzoyl]}cyclooctanol 13

Under the protection of nitrogen, a solution of 4-bromo-N,N-(dihexyl)aniline 3a (12.7 g, 0.37 mmol) in dry THF (20 ml) was added dropwise at room temperature to a stirred mixture of magnesium turnings (1 g, 41 mol), 5 ml dry THF and two drops of 1,2-dibromoethane. The addition was finished in half an hour and stirring was maintained for two more hours at room temperature. A solution of 12 (7.1 g, 31.1 mmol) in 10 ml dry THF was added dropwise to the solution of the Grignard reagent and the mixture was stirred at reflux for 48 hours. After this time 26 ml 6 N HCl was carefully added into the mixture with ice cooling and vigorous stirring. The mixture was then stirred at room temperature for 4 more hours and then sodium bi-carbonate was used to neutralize the excess acid and the solid in the mixture was removed by vacuum filtration through a pad of Celite. The filtrate was then extracted with EtOAc and after drying the organic solution over anhydrous MgSO₄ and evaporation of the solvent, the crude product was purified by column chromatography (solvent: EtOAc/hexane=1/9) to give 6.2 g (yield 40%) of clear liquid: ¹H NMR (300 MHz, CDCl₃) δ 0.89 (t, J=6.9 Hz, 6 H), 1.31-2.41 (m, 30 H), 3.31 (t, J=7.8 Hz, 4 H), 4.32 (s, 1 H), 6.58 (d, J=9.3 Hz, 2 H), 8.02 (d, J=9.3 Hz, 2 H); ¹³C NMR (75 MHz, CDCl₃) δ 14.14, 21.47, 22.78, 24.11, 26.85, 27.31, 27.96, 31.79, 36.44, 51.10, 79.33, 110.14, 119.68, 133.00, 151.48, 202.05 ppm; IR (neat, cm⁻¹) 3409 (OH), 1590 (C═O).

### Example 19

### Preparation of 2-butyl-2-trimethylsilyloxyhexylcarbonitrile 14

Using a method identical to that described for 12, TMSCN (4.1 g, 41.3 mmol) reacted with nonan-5-one (5.35 g, 37.6 mmol) to give a clear liquid (8.7 g, 96%) as product: ¹H NMR (300 MHz, CDCl₃) δ 0.23 (s, 9 H), 0.93 (t, J=7.2 Hz, 6 H), 1.40 (m, 8 H), 1.71 (t, J=8.4 Hz, 4 H); ¹³C NMR (75 MHz, CDCl₃) δ 1.473, 14.05, 22.71, 26.27, 40.87, 73.37, 121.94 ppm.

### Example 20

### Preparation of 2-butyl-1-{4-[N,N-(dihexyl)aminophenyl]}-2-hydroxyhexan-1-one 15

In the same way described already for 13, starting from 3a (6.26 g, 18.4 mmol) in 10 ml THF, magnesium (0.49 g, 20.2 mmol) in 3 ml THF and 14 (3.7 g, 15.3 mmol) in 10 ml THF, a clear liquid (3.7 g, 47%) was obtained: ¹H NMR (300 MHz, CDCl₃) δ 0.81 (t, J=7.2 Hz, 6 H), 0.91 (t, J=6.9 Hz, 6 H), 1.21-2.2 (m, 28 H), 3.33 (t, J=7.8 Hz, 4 H), 4.87 (s, 1 H), 6.58 (d, J=9.0 Hz, 2 H), 7.96 (d, J=9.0 Hz, 2 H); ¹³C NMR (75 MHz, CDCl₃) δ 14.09, 14.21, 22.84, 23.24, 25.69, 26.92, 27.36, 31.82, 41.37, 51.16, 80.74, 110.30, 120.30, 132.20, 151.80, 201.04 ppm.

### Example 21

### Preparation of 3-cyano-2-dicyanomethylen-4-{4″-[N,N-(dihexyl)aminophenyl]}-1-oxaspiro[4,7]dodec-3-ene (Entry 12, DCDHF-6-C7M)

A mixture of 13 (5.8 g, 14 mmol), malononitrile (4.48 g, 68 mmol) and pyridine (40 ml) was stirred at 80-90° C. for 24 hours under the protection of dry nitrogen. After the reaction, 300 ml water was added and the mixture was extracted with ethyl acetate. The organic layer was washed with water several times to remove the pyridine, dried over magnesium sulfate and concentrated in vacuo. The crude product was purified by column chromatography (solvent: ethyl acetate/hexane=1/9) and recrystallized from CH₂Cl₂/methanol to give 2.7 g (38%) of red crystals: mp 150° C. ¹H NMR (300 MHz, CDCl₃) δ 0.89 (t, J=6.6 Hz, 6 H), 1.33 (m, 12 H), 1.63 (m, 12 H), 1.94 (m, 2 H), 2.08 (m, 2 H), 2.24 (m, 2 H), 3.39 (d, J=7.2 Hz, 4 H), 6.70 (d, J=9.3 Hz, 2 H), 8.01 (d, J=9.3 Hz, 2 H); ¹³C NMR (75 MHz, CDCl₃) δ 14.17, 21.68, 22.77, 23.40, 26.80, 27.25, 27.47, 31.72, 36.82, 51.58, 53.68, 89.77, 101.22, 112.10, 112.46, 113.39 (2 carbons), 113.65, 133.00, 152.90, 176.67, 177.80 ppm; IR (neat, cm⁻¹) 2953.62, 2925.42, 2856.71, 2221.09, 2208, 1607.75, 1564.03, 1541.06, 1353.68, 1184.44, 1111.06, 1017.25.

### Example 22

### Preparation of 5,5-dibutyl-3-cyano-2-dicyanomethylen-4-{4″-[N,N-(dihexyl)aminophenyl]}-2,5-dihydrofuran (Entry 13, DCDHF-6-DB)

In the same way described already for DCDHF-6-C7M, starting from 15 (0.51 g, 1.2 mmol) and malononitrile (0.5 g, 7.6 mmol), yellow crystals (0.125 g, 20%) were obtained as product: mp 95° C. ¹H NMR (300 MHz, CDCl₃) δ 0.85 (t, J=7.1 Hz, 6 H), 0.92 (t, J=6.9 Hz, 6 H), 1.20-2.18 (m, 28 H), 3.39 (t, J=7.8 Hz, 4 H), 6.68 (d, J=9.3 Hz, 2 H), 7.95 (d, J=9.3 Hz, 2 H); ¹³C NMR (75 MHz, CDCl₃) δ 13.90, 14.16, 22.58, 22.78, 25.00, 26.83, 27.47, 31.72, 40.06, 51.53, 53.43, 93.03, 103.21, 112.06, 112.35, 113.23, 113.36, 113.85, 131.90, 152.97, 172.13, 178.20 ppm.

### Example 23

### Preparation of 1-(4-fluorophenyl)-2-trifluoromethyl-2-hydroxypropan-1-one 18

A mixture of TMSCN (5 g, 50.4 mmol), 1,1,1-trifluoroacetone (6.8 g, 60.7 mmol, low boiling point, chill before handling) and dry THF (50 ml) was stirred in a flame-dried flask with external ice bath cooling. A catalytic amount of n-BuLi (2.5 M in hexanes, 0.2 ml) was added with a syringe at 0° C. After stirring at room temperature for 4 hours, house vacuum was applied on the mixture to remove any excess 1,1,1-trifluoroacetone. In a second flask, under the protection of nitrogen, a solution of 4-bromofluorobenzene (21.6 g, 0.123 mol) in dry THF (40 ml) was added drop-wise at room temperature to a stirred mixture of magnesium turnings (2.5 g, 0.103 mol), 10 ml dry THF and one drop of 1,2-dibromoethane. An ice water bath was occasionally used to moderate the reaction temperature. The addition was finished in half an hour and stirring was maintained for one more hour at room temperature. At room temperature, the clear solution in the first flask was then transferred to the second flask containing the Grignard via a dry syringe. The addition is slightly exothermic and can be detected by hand. After five hours, 68 ml 6 N HCl was carefully added into the mixture with ice cooling and vigorous stirring. The mixture was then stirred at room temperature for 2 more hours until TLC showed only one major spot and then sodium bicarbonate was used to neutralize the excess acid and the solid in the mixture was removed by vacuum filtration through a pad of Celite. The filtrate was then extracted with EtOAc and after drying the organic solution over anhydrous MgSO₄ and evaporation of the solvent, the liquid product was purified by column chromatography (solvent: EtOAc/hexane=1/9) to give 11.64 g (yield 98%) of clear liquid: ¹H NMR (300 MHz, CDCl₃) δ 1.77 (s, 3 H), 4.79 (s, 1 H), 7.12 (dd, J=9.0, 8.4 Hz, 2 H), 8.13 (dd, J=9.0, 5.4 Hz, 2 H); ¹³C NMR (75 MHz, CDCl₃) δ 20.77, 79.2-80.3 (q, J=28.3 Hz), 115.5 (d, J=21.7 Hz), 118.6-129.9 (q, J=284.3 Hz), 130.48, 133.3 (d, J=9.3 Hz), 166.0 (d, J=254.9 Hz), 195.19 ppm; ¹⁹F NMR (282 MHz, CDCl₃) δ− 103.14 (tt, J=8.4, 5.4 Hz, 1 F), −77.82 (s, 3 F).

### Example 24

### Preparation of 2-trifluoromethyl-1-{4″-[N,N-(dihexyl)aminophenyl]}-2-hydroxy-propan-1-one 19b

A mixture of dihexylamine (8.2 g, 44.5 mmol), p-TsOH (0.15 g, 0.79 mmol), 18 (3.5 g, 14.8 mmol) and DMSO (15 g) was stirred at 165° C. for 14 hours. After the reaction, DMSO and excess dihexylamine was removed by Kugelrohr distillation and ethyl acetate were then added to the remaining crude product. The ethyl acetate solution was filtered through a short pad of silica gel and then concentrated to give 5.77 g (yield 97%) of product as a viscous oil: ¹H NMR (300 MHz, CDCl₃) δ 0.90 (t, J=6.6 Hz, 6 H), 1.33 (m, 12 H), 1.61 (m, 4 H), 1.82 (s, 3 H), 3.34 (t, J=7.8 Hz, 4 H), 5.44 (s, br, 1 H), 6.59 (d, J=9.0 Hz, 2 H), 8.01 (d, J=9.0 Hz, 2 H); ¹³C NMR (75 MHz, CDCl₃) δ 14.16, 21.73, 22.79, 26.85, 27.31, 31.69, 51.21, 76.99-78.30 (q, J=29.1 Hz), 110.42, 118.78, 118.89-130.14 (q, J=283.5 Hz), 133.87, 152.55, 191.69 ppm; ¹⁹F NMR (282 MHz, CDCl₃) δ −77.76 (s, 3 F); IR (neat, cm⁻¹) 1588, 1661, 3369.

### Example 25

### Preparation of 1-[4-(azepan-1-yl)phenyl]-2-trifluoromethyl-2-hydroxypropan-1-one 19c

Using the same method just described for 19b, starting with azepane (OLE_LINK6hexamethyleneimineOLE_LINK6) (4.2 g, 42.4 mmol), a few crystals of p-TsOH, 18 (3.3 g, 14 mmol) and DMSO (7 g), a clear viscous oil (4.3 g, 98%) was obtained: ¹H NMR (300 MHz, CDCl₃) δ 1.55 (m, 4 H), 1.80 (m, 4 H), 1.82 (s, 3 H), 3.53 (t, J=6.0 Hz, 4 H), 5.41 (s, 1 H), 6.65 (d, J=9.3 Hz, 2 H), 8.01 (d, J=9.3 Hz, 2 H); ¹³C NMR (75 MHz, CDCl₃) δ 21.72, 26.84, 27.31, 49.64, 77.16-78.34 (q, J=29.48 Hz), 110.345, 119.04, 118.80-130.14 (q, J=283.5 Hz), 133.94, 153.34, 191.76 ppm; ¹⁹F NMR (282 MHz, CDCl₃) δ −77.76 (s, 3 F); IR (neat, cm⁻¹) 1584 (C═O), 3380 (OH).

### Example 26

### Preparation of 1-(4-diethylaminophenyl)-2-hydroxy-2-trifluoromethyl-propan-1-one 19a

Using the same method just described for 19b, starting with diethylamine (10 g, 0.137 mol), p-TsOH (0.064 g, 0.34 mmol), 18 (4 g, 17 mmol) and DMSO (15 ml) in a Fischer-Porter bottle, a clear liquid (4.8 g, 98%) was obtained: ¹H NMR (300 MHz, CDCl₃) δ 1.17 (t, J=7.1 Hz, 6 H), 1.77 (s, 3 H), 3.39 (q, J=7.1 Hz, 4 H), 5.52 (s, 1 H), 6.59 (d, J=9.3 Hz, 2 H), 8.01 (d, J=9.3 Hz, 2 H); ¹³C NMR (75 MHz, CDCl₃) δ 12.52, 21.66, 44.74, 77.40-78.55 (q, J=29.3 Hz), 110.26, 119.05, 118.91-130.25 (q, J=285.5 Hz), 133.90, 152.12, 191.91 ppm; ¹⁹F NMR (282 MHz, CDCl₃) δ −77.76 (s, 3 F); IR (neat, cm⁻¹) 3361, 2978, 1646, 1585.

### Example 27

### Preparation of 3-cyano-2-dicyanomethylen-5-trifluoromethyl-4-{4″-[N,N-(dihexyl)aminophenyl]}-5-methyl-2,5-dihydrofuran (Entry 15, DCDHF-6-CF3)

A mixture of 19b (5.77 g, 14.4 mmol), pyridine (60 ml) and 5 drops of acetic acid was stirred at 130° C. under the protection of dry nitrogen. A mixture of malononitrile (4.75 g, 72 mmol) and pyridine (30 ml) was added to the reaction flask via a dropping funnel in 3 portions within 3 hours. Eight hours later, the reaction was stopped and the reaction mixture was extracted with ethyl acetate and water. The organic layer was washed with water several times to remove the pyridine. After drying the organic solution over anhydrous MgSO₄ and evaporation of the solvent, the mixture was purified by column chromatography (solvent: CH₂Cl₂/hexane=1/1). The product was recrystallized from ethanol to give 1.95 g (yield 27%) black metallic crystals, mp 130.4° C. ¹H NMR (300 MHz, CDCl₃) δ 0.90 (t, J=6.6 Hz, 6 H), 1.34 (m, 12 H), 1.65 (m, 4 H), 2.09 (s, 3 H), 3.43 (t, J=7.8 Hz, 4 H), 6.71 (d, J=9.3 Hz, 2 H), 8.00 (d, J=9.3 Hz, 2 H); ¹³C NMR (75 MHz, CDCl₃) δ 14.14, 21.07, 22.75, 26.78, 27.54, 31.67, 51.73, 55.94, 92.61, 93.94-95.23 (q, J=32.1 Hz), 111.18, 111.88, 112.40, 112.78, 113.24, 116.69-128.06 (q, J=284.3 Hz), 133.41, 153.63, 163.52, 176.61 ppm; ¹⁹F NMR (282 MHz, CDCl₃) δ −77.66 (s, 3 F); IR (neat, cm⁻¹) 2224 (CN).

### Example 28

### Preparation of 3-cyano-2-dicyanomethylen-4-{4″-[N,N-(diethyl)aminophenyl]}-5-trifluoromethyl-5-methyl-2,5-dihydrofuran (Entry 16, DCDHF-2-CF3)

In the same way as just described for DCDHF-6-CF3, starting with 19a (4.9 g, 16.9 mmol), acetic acid (4 drops) and malononitrile (2.24 g, 33.9 mmol), black metallic crystals (0.96 g, 15%) were obtained: mp 180° C. ¹H NMR (300 MHz, CDCl₃) δ 1.28 (t, J=7.1 Hz, 6 H), 2.09 (s, 3 H), 3.53 (q, J=7.1 Hz, 4 H), 6.76 (d, J=9.3 Hz, 2 H), 8.00 (d, J=9.3 Hz, 2 H); ¹³C NMR (75 MHz, CDCl₃) δ 12.82, 21.04, 45.56, 56.49, 93.20, 93.87-95.16 (q, J=32 Hz), 111.18, 111.89, 112.31, 112.73, 113.32, 116.68-128.05 (q, J=284.4 Hz), 133.50, 153.29, 163.72, 176.33 ppm; ¹⁹F NMR (282 MHz, CDCl₃) δ −77.66 (s, 3 F).

### Example 29

### Preparation of 4-[4-(azepan-1-yl)phenyl]-3-cyano-2-dicyanomethylen-5-trifluoromethyl-5-methyl-2,5-dihydrofuran (Entry 14, DCDHF-C6M-CF3)

In the same way as just described for DCDHF-6-CF3, starting with 19c (4.3 g, 13.6 mmol), malononitrile (3.6 g, 54.5 mmol), acetic acid (0.8 mg) and pyridine (40 ml), black metallic crystals (0.5 g, 10%) were obtained as product: mp 214° C. (from EtOAc/Methanol); ¹H NMR (300 MHz, CDCl₃) δ 1.59 (m, 4 H), 1.84 (m, 4 H), 2.09 (s, 3 H), 3.64 (t, J=6.0 Hz, 4 H), 6.79 (d, J=9.3 Hz, 2 H), 8.00 (d, J=9.3 Hz, 2 H); ¹³C NMR (75 MHz, CDCl₃) δ 21.06, 26.55, 27.11, 50.48, 56.14, 92.83, 93.88-95.17 (q, J=32.3 Hz), 111.30, 112.00, 112.36, 112.83, 113.44, 116.71-128.08 (q, J=284.4 Hz), 133.58, 154.43, 163.60, 176.44 ppm; ¹⁹F NMR (282 MHz, CDCl₃) δ −77.66 (s, 3 F); IR (neat, cm⁻¹) 2225.

### Example 30

### Preparation of N,N-dihexyl-4-formylaniline 22c

Two steps procedure starting from aniline was used for preparing the title compound.

N,N-dihexylaniline was synthesized from a mixture of aniline, 1-bromohexane and potassium hydroxide: clear liquid; ¹H NMR (300 MHz, CDCl₃) δ 0.92 (t, J=6.6 Hz, 6 H), 1.33 (m, 12 H), 1.59 (m, 4 H), 3.26 (t, J=7.8 Hz, 4 H), 6.64 (m, 3 H), 7.22 (m, 2 H); ¹³C NMR (75 MHz, CDCl₃) δ 14.24, 22.89, 27.06, 27.38, 31.94, 51.23, 111.81, 115.19, 129.35, 148.34.

Phosphorous oxychloride (9.9 ml, 106 mmol) was added dropwise to stirred dry DMF (26.2 ml, 338 mmol) at 0° C. The resulting red mixture was kept stirring at this temperature for 30 minutes and then N,N-dihexylaniline (25.23 g, 96.5 mmol) was added to the mixture at 0° C. The resulting solution was then heated at 90° C. for 4 hours. After this time, water (400 ml) was slowly and carefully added to the mixture at room temperature. The acid produced in the mixture was neutralized by careful addition of solid sodium bicarbonate. The resulting mixture was extracted with ethyl acetate and the organic layer was washed with water, dried over magnesium sulfate, concentrated and flash chromatographed to give a clear oil (24.3 g, 87%): ¹H NMR (300 MHz, CDCl₃) δ 0.90 (t, J=6.8 Hz, 6 H), 1.32 (m, 12 H), 1.60 (m, 4 H), 3.33 (t, J=7.7 Hz, 4 H), 6.63 (d, J=9.0 Hz, 2 H), 7.68 (d, J=9.0 Hz, 2 H), 9.69 (s, 1 H); ¹³C NMR (75 MHz, CDCl₃) δ 14.71, 22.79, 26.87, 27.27, 31.79, 51.27, 110.81, 124.64, 132.36, 152.75, 190.07 ppm.

### Example 31

### Preparation of N,N-[di-(2-ethylhexyl)]-4-formylaniline 22d

Three steps procedure starting from 4-bromoaniline was used to prepare the title compound.

A mixture of 15 g (87.2 mmol) of 4-bromoaniline, 20 g (104.6 mmol) 2-ethylhexylbromide, 36.7 g (262 mmol), 2 g of potassium iodide and 2 g of tetrabutylamonium chloride in 100 ml of DMF was heated under reflux during 12 h. The mixture obtained was extracted with EtOAc/H₂O and dried with Na₂SO₄. After purification by vacuum distillation, 15.5 g (63% yield) of a yellow oil was obtained. Part of this oil, 3 g (10.6 mmol) was dissolved in 30 ml of dry THF and 8 ml (20 mmol) of 2.5 M n-BuLi in hexanes was added at 78° C. After stirring for 1 h at 78° C., 3 g (15.5 mmol) of 2-ethylhexylbromide in 20 ml dry THF was added and the stirring was maintained at this temperature for one more hour. The mixture was allowed to warm to room temperature overnight, hydrolyzed with 5 N HCl with ice cooling, and diluted with CHCl₃. The aqueous phase was removed and extracted with CHCl and the combined organic phases were washed with H₂O, dried (NaSO₄) and evaporated in vacuo. Silica gel column chromatography using petroleum ether as eluent gave 2.5 g (71% yield) of the product, 4-bromo-N,N-[di(2-ethylhexyl)]aniline, as a yellow oil: ¹H-NMR (300 MHz, CDCl₃) δ 7.23 (d, J=9.0 Hz, 2H), 6.52 (d, J=9.0 Hz, 2H), 3.16 (m, 4H), 1.73 (m, 2 H), 1.24 (m, 16 H), 0.88 (m, 12H).

To a solution of the above 4-bromo-N,N-[di(2-ethylhexyl)]aniline in dry THF (100 ml) was added dropwise 3.7 ml (9 mmol) 2.5 M n-BuLi in hexane over 30 min at 78° C. After stirring for 1 h at 78° C., 0.75 ml (9 mmol) of dry dimethylformamide was added in one portion. After stirring for one additional hour at 78° C., the mixture was allowed to warm to room temperature overnight, then hydrolyzed with 5 N HCl (1.85 ml) with ice cooling and diluted with CHCl₃. The aqueous phase was extracted with CHCl₃ and the combined organic phases were washed with H₂O, dried (NaSO₄) and evaporated in vacuo. Purification under silica gel column chromatography using petroleum ether: ethyl acetate (20:1) gave 2.5 g (99% yield) of the product, N,N-[di-(2-ethylhexyl)]-4-formylaniline, as a yellow oil: ¹H-NMR (300 MHz, CDCl₃) δ 9.71 (s, 1 H), 7.70 (d, J=9.0 Hz, 2 H), 6.68 (d, J=9.0 Hz, 2 H), 3.31 (m, 4 H), 1.83 (m, 2 H), 1.28 (m, 16 H), 0.90 (m, 12 H); ¹³C-NMR (75 MHz, CDCl₃) δ 190.04, 152.96, 132.09, 124.68, 111.77, 56.65, 37.05, 30,85, 28.77, 24.04, 23.28, 14.21, 10.83 ppm.

### Example 32

### Preparation of 4,4″-di(carbazol-9-yl)-4″-{6-[N-ethyl-N-(4-formylphenyl)amino]}hexyloxytriphenyl-amine 22 g

The title aldehyde was synthesized according to literature procedure (He, M.; Twieg, R. J.; Gubler, U.; Wright, D.; Moerner, W. E. “Synthesis and Photorefractive Properties of Multifunctional Glasses,” Chem. Mater. 2003, 15, 5, 1156-1164).

### Example 33

### Preparation of 3-cyano-2-dicyanomethylen-4,5,5-trimethyl-2,5-dihydrofuran 21

A mixture of 92% 3-hydroxy-3-methylbutan-2-one 20 (9.5 g, 85.6 mmol), malononitrile (12.3 g, 186 mol), two drops of acetic acid and pyridine (50 ml) was stirred at room temperature for 24 hours. The reaction temperature was controlled without exceeding the room temperature by the use of an ice bath at the beginning of the reaction.

The reaction mixture was then poured into 800 ml ice water with vigorous stirring. The precipitate was collected by vacuum filtration and recrystallized from ethanol to give 13.6 g (80% yield) of white crystals: mp 203° C. (lit. 199° C., Melikian, G.; Rouessac, F. P.; Alexandre, C. Synth. Commun. 1995, 25, 19, 3045). ¹H NMR (300 MHz, CDCl₃) δ 1.61 (s, 6 H), 2.35 (s, 3 H); ¹³C NMR (75 MHz, CDCl₃) δ 14.26, 24.47, 58.65, 99.87, 104.98, 109.07, 110.51, 111.11, 175.30, 182.63 ppm.

### Example 34

### Preparation of 1-(3-cyano-2-dicyanomethylen-5,5-dimethyl-2,5-dihydrofuran-4-yl)-2-{4-[N,N-(di-(2-ethylhexyl))aminophenyl]}ethene (Entry 23, DCDHF-2EH-V)

A mixture of 2 g (6 mmol) of 4-bis-(2″-ethylhexylamino)-benzaldehyde, 715 mg (6 mmol) of 3-cyano-2-dicyanomethylen-4,5,5-trimethyl-2,5-dihydrofuran 21 and 320 mg of acetic acid were dissolved in 20 ml of dry pyridine. After addition of 1 g of 3 Å molecular sieves the mixture was stirred overnight at room temperature. Pyridine was distilled out under vacuum and the blue syrup obtained was purified by silica gel column chromatography using petroleum ether: ethyl acetate (1:1) as eluent. After recrystallization from methanol 1.8 g (57% yield) of the product was obtained as green crystals: mp 100° C.; ¹H-NMR (300 MHz, CDCl₃) δ 7.60 (d, J=15.6, 1 H), 7.51 (d, J=9.0, 2 H), 6.72 (d, J=15.6, 1 H), 6.69 (d, J=9.0 Hz, 2 H), 3.33 (m, 4 H), 1.80 (m, 2 H), 1.74 (s, 6 H), 1.28 (m, 16 H), 0.90 (m, 12 H); ¹³C-NMR (75 MHz, CDCl₃) δ 10.85, 14.23, 23.24, 24.04, 26.93, 28.78, 30.70, 37.54, 53.86, 56.57, 93.26, 96.97, 108.33, 111.91, 112.41, 113.15, 113.20, 121.63, 132.71, 148.82, 152.85, 174.51, 176.71 ppm.

### Example 35

### Preparation of 1-(3-cyano-2-dicyanomethylen-5,5-dimethyl-2,5-dihydrofuran-4-yl)-2-{4-[N,N-(diethyl)aminophenyl]}ethene (Entry 20, DCDHF-2-V)

A mixture of N,N-diethyl-4-formylaniline (1.5 g, 8.46 mmol),21 (0.9 g, 4.52 mmol), acetic acid (0.04 g) and pyridine (15 ml) was stirred at room temperature for 24 hours. The reaction mixture was then poured into 300 ml water, the precipitate collected was recrystallized from CH₂Cl₂/methanol to give the product as black crystals (1.4 g, 88%): mp 245 ° C. ¹H NMR (300 MHz, CDCl₃) δ 1.24 (t, J=7.1 Hz, 6 H), 1.74 (s, 6 H), 3.48 (q, J=7.1 Hz, 4 H), 6.68 (d, J=9.0 Hz, 2 H), 6.71 (d, J=15.6 Hz, 1 H), 7.53 (d, J=9.0 Hz, 2 H), 7.62 (d, J=15.6 Hz, 1 H); ¹³C NMR (75 MHz, CDCl₃) δ 12.75, 26.88, 45.14, 54.15, 93.53, 96.91, 108.34, 111.79, 112.22 (2 carbons), 112.98, 121.68, 132.89, 148.72, 152.22, 174.50, 176.61 ppm.

### Example 36

### Preparation of 9-[2-(3-cyano-2-dicyanomethylen-5,5-dimethyl-2,5-dihydrofuran-4-yl)vinyl]-2,3,6,7-tetrahydro-1H, 5H-pyrido[3,2,1-ij]quinoline (entry 21, DCDHF-J-V)

In the same way described already for DCDHF-2-V, starting with a mixture of 2,3,6,7-tetrahydro-1H,5H-pyrido[3,2,1-ij]quinoline-9-carbaldehyde 22b (0.505 g, 2.5 mmol),3-cyano-2-dicyanomethylen-4,5,5-trimethyl-2,5-dihydrofuran 21 (0.5 g, 2.5 mmol), acetic acid (0.04 g) and pyridine (10 ml), metallic green crystals were obtained (0.4 g, yield 42%): mp 243° C. ¹H NMR (300 MHz, CDCl₃) δ 1.72 (s, 6 H), 1.99 (m, 4 H), 2.77 (t, J=6.3 Hz, 4 H), 3.39 (t, J=5.8 Hz, 4 H), 6.64 (d, J=15.7 Hz, 1 H), 7.13 (s, 2 H), 7.52 (d, J=15.7 Hz, 1 H).

### Example 37

### Preparation of 1-(3-cyano-2-dicyanomethylen-5,5-dimethyl-2,5-dihydrofuran-4-yl)-2-{4-[N,N-(diphenylaminophenyl)]}ethene (entry 21, DCDHF-DPH-V)

In the same way described already for DCDHF-2-V, starting with a mixture of 4-diphenylaminobenzaldehyde (0.87 g, 3.2 mmol), 3-cyano-2-dicyanomethylen-4,5,5-trimethyl-2,5-dihydrofuran 21 (0.58 g, 2.91 mmol), acetic acid (0.04 g) and pyridine (15 ml), black crystals (1.12 g, 85%) were obtained: mp 330.5° C. ¹H NMR (300 MHz, CDCl₃) δ 1.77 (s, 6 H), 6.82 (d, J=15.9 Hz, 1 H), 6.99 (d, J=9.0 Hz, 2 H), 7.17-7.23 (m, 6 H), 7.34-7.40 (m, 4 H), 7.47 (d, J=9.0 Hz, 2 H), 7.59 (d, J=15.9 Hz, 1 H); ¹³C NMR (75 MHz, CDCl₃) δ 26.65, 56.33, 96.86, 97.09, 110.86, 111.22, 111.34, 112.14, 119.94, 125.66, 125.97, 126.39, 129.87, 131.09, 145.72, 147.20, 152.56, 173.92, 175.79.

### Example 38

### Preparation of 1-(3-cyano-2-dicyanomethylen-5,5-dimethyl-2,5-dihydrofuran-4-yl)-2-{4-[N,N-(dihexylaminophenyl)]}ethene (Entry 22, DCDHF-6-V)

In the same way described already for DCDHF-2-V, a mixture of N,N-dihexyl-4-formylaniline (2.23 g, 7.7 mmol), 21 (1.46 g, 7.32 mmol), acetic acid (5 drops) and pyridine (20 ml) was reacted to give black crystals (3.07 g, yield 89%) as the product: mp 147° C. ¹H NMR (300 MHz, CDCl₃) δ 0.90 (t, J=6.6 Hz, 6 H), 1.33 (m, 12 H), 1.63 (m, 4 H), 1.74 (s, 6 H), 3.39 (t, J=7.8 Hz, 4 H), 6.66 (d, J=9.0 Hz, 2 H), 6.70 (d, J=15.6 Hz, 1 H), 7.53 (d, J=9.0 Hz, 2 H), 7.63 (d, J=15.6 Hz, 1 H); ¹³C NMR (75 MHz, CDCl₃) δ 14.19, 22.77, 26.82, 26.94, 27.49, 31.73, 51.51, 53.87, 93.19, 96.93, 108.19, 111.93, 112.36 (2 carbons, one CN was buried inside), 113.16, 121.56, 132.93, 148.76, 152.62, 174.44, 176.68 ppm.

### Example 39

### Preparation of 1-(3-cyano-2-dicyanomethylen-5,5-dimethyl-2,5-dihydrofuran-4-yl)-2-(4-{N,N-[di(2-methoxylethyl)]}aminophenyl)ethene (Entry 24, DCDHF-MOE-V)

In the same way just described for DCDHF-2-V, starting with a mixture of 4-formyl-N,N-[di-(2-methoxyethyl)]aniline (1.3 g, 5.5 mmol), 21 (0.5 g, 2.5 mmol), acetic acid (0.02 g) and pyridine (20 ml), black crystals (0.85 g, 81%) were obtained: mp 212° C. ¹H NMR (300 MHz, CDCl₃) δ 1.74 (s, 6 H), 3.35 (s, 6 H), 3.59 (t, J=5.4 Hz, 4 H), 3.70 (t, J=5.4 Hz, 4 H), 6.72 (d, J=15.9 Hz, 1 H), 6.77 (d, J=8.7 Hz, 2 H), 7.52 (d, J=8.7 Hz, 2 H), 7.62 (d, J=15.9 Hz, 1 H); ¹³C NMR (75 MHz, CDCl₃) δ 26.86, 51.38, 54.77, 59.26, 70.15, 94.45, 96.98, 109.00, 111.57, 112.80, 112.90, 113.01, 122.30, 132.48, 148.47, 152.77, 174.46, 176.42 ppm.

### Example 40

### Preparation of 2-[4-(2-{4-[(6-{4-[bis-(4-carbazol-9-ylphenyl)amino]phenoxy}hexyl)ethylamino]-phenyl}vinyl)-3-cyano-5,5-dimethyl-5H-furan-2-ylidene]malononitrile DCTA-DCDHF 124

A mixture of 4,4″-di(carbazol-9-yl)-4″-{6-[N-ethyl-N-(4-formylphenyl)amino]}hexyloxytriphenyl-amine 22g (0.90 g, 1.1 mmol), 21 (0.22 g, 1.1 mmol), acetic acid (one drop) and pyridine (15 ml) was stirred at room temperature for 48 hours. The reaction mixture was then poured into water (200 ml) and the precipitate was collected. Flash chromatography on silica gel was used (CHCl₃/hexane=1/1) to purify the product and the solid obtained was further purified by dissolution in CH₂Cl₂ and precipitation from methanol to give the product as a purple powder (0.85 g, 77% yield): T 141° C.; ¹H NMR (300 MHz, CDCl₃) δ 1.27 (t, J=6.9 Hz, 3 H), 1.46-1.82 (m, 8 H), 1.77 (s, 6 H), 3.41-3.53 (m, 4 H), 4.04 (t, J=6.1 Hz, 2 H), 6.7-7.7 (m, 30 H), 8.19 (d, J=7.5 Hz, 4 H); ¹³C NMR (75 MHz, CDCl₃) δ 12.67, 26.27, 26.96, 27.08, 27.75, 29.55, 45.86, 50.93, 54.27, 68.21, 93.73, 96.99, 108.53, 110.07, 111.61, 111.92, 112.43, 113.16, 115.96, 120.04, 120.54, 121.85, 123.44, 123.72, 126.12, 128.15, 128.39, 131.59, 132.94, 140.09, 141.28, 147.22, 148.61, 152.27, 156.71, 174.44, 176.65 ppm; IR (neat, cm⁻¹) 2933, 2224, 1596, 1559, 1504, 1451; UV-Vis (THF) 572 (ε=67000 L mol⁻¹ cm⁻¹).

### Example 41

### Preparation of 1-(5-bromothiophen-2-yl)-2-hydroxy-2-methylpropan-1-one 25

Under the protection of nitrogen, a solution of 2,5-dibromothiophene (43.8 g, 0.172 mol) in dry THF (100 ml) was added dropwise at room temperature to a stirred mixture of magnesium turnings (3.86 g, 0.16 mol) in 20 ml of dry THF. An ice water bath was occasionally used to moderate the reaction temperature. The addition was finished in half an hour and stirring was maintained for four more hours at room temperature and then 2 hours under refluxing until the magnesium was consumed. A solution of 2-methyl-2-trimethylsilyloxypropionitrile 5 (25 g, 0.16 mol) in 50 ml dry THF was added to the solution of the Grignard reagent and the mixture was stirred at 90° C. for 24 hours. After this time 160 ml 6 N HCl was carefully added into the mixture with ice cooling and vigorous stirring. The mixture was then stirred at room temperature for 4 more hours and then sodium bicarbonate was used to neutralize the excess acid and the solid in the mixture was removed by vacuum filtration through a pad of Celite. The filtrate was extracted with EtOAc and after drying the organic solution over anhydrous MgSO and evaporation of the solvent, the crude product was purified by column chromatography (solvent: EtOAc/hexane=1/9) to give 14.1 g (yield 36%) yellow oil: ¹H NMR (300 MHz, CDCl₃) δ 1.57 (s, 6 H), 3.40 (s, 1 H), 7.11 (d, J=4.0 Hz, 1 H), 7.70 (d, J=4.0 Hz, 1 H); ¹³C NMR (75 MHz, CDCl₃) δ 28.40, 76.92, 123.97, 131.16, 135.07, 140.03, 196.00 ppm.

### Example 42

### Preparation of 1-[5-(N,N-di-hexyl)aminothien-2-yl]-2-hydroxy-2-methylpropan-1-one 26a

A mixture of 25 (8.08 g, 32.4 mmol), dihexylamine (18 g, 97.1 mmol), p-TsOH (0.12 g, 0.63 mmol) and DMSO (30 ml) was stirred at 170° C. for 12 hours. After this time, most of the DMSO and dihexylamine were removed by Kugelrohr distillation and the crude residue was purified by column chromatography (solvent: EtOAc/hexane=1/9) to give 4.5 g (yield 39%) of the product as a viscous yellow oil: ¹H NMR (300 MHz, CDCl₃) δ 0.86 (t, J=6.6 Hz, 6 H), 1.30 (m, 12 H), 1.58 (s, 6 H), 1.61 (m, 4 H), 3.31 (t, J=7.7 Hz, 4 H), 4.57 (s, br, 1 H), 5.83 (d, J=4.5 Hz, 1 H), 7.55 (d, J=4.5 Hz, 1 H); ¹³C NMR (75 MHz, CDCl₃) δ 14.14, 22.69, 26.70, 26.99, 29.69, 31.65, 53.85, 74.75, 102.96, 120.04, 137.77, 166.63, 193.43 ppm; IR (neat, cm⁻¹) 1581 (C═O), 3459 (OH).

### Example 43

### Preparation of 1-[5-(azepan-1-yl)thieny-2-yl]-2-hydroxy-2-methylpropan-1-one 26b

A mixture of 25 (3.2 g, 12.8 mmol), azepane (hexamethyleneimine) (3.8 g, 38.3 mmol) and p-TsOH (0.12 g, 0.63 mmol) was stirred at 120° C. for 24 hours. After this time, water (15 ml) and petroleum ether (15 ml) were added and the mixture was stirred at room temperature for 20 minutes. The precipitated solids were then collected by vacuum filtration and recrystallized from CH₂Cl₂/EtOAc to give 2.7 g (yield 79%) of the product as yellow crystals: mp 120° C. ¹H NMR (300 MHz, CDCl₃) δ 1.59 (s, 6 H), 1.61 (m, 4 H), 1.83 (m, 4 H), 2.99 (s, br, 1 H), 3.50 (t, J=5.9 Hz, 4 H), 5.88 (d, J=4.2 Hz, 1 H), 7.61 (d, J=4.2 Hz, 1 H); ¹³C NMR (75 MHz, CDCl₃) δ 27.38, 27.46, 29.76, 52.57, 74.65, 102.37, 119.95, 137.80, 167.05, 193.50 ppm; IR (neat, cm⁻¹) 1581 (C═O), 3407 (OH).

### Example 44

### Preparation of 3-cyano-2-dicyanomethylen-5,5-dimethyl-4-[5-(N,N-dihexyl)aminothien-2-yl]-2,5-dihydrofuran (Entry 17, TH-DCDHF-6)

A mixture of 26a (1.7 g, 4.8 mmol), malononitrile (1.27 g, 19.2 mmol), acetic acid (0.58 g, 10 mmol), 2 g 3 Å molecular sieves and pyridine (20 ml) was stirred at 90° C. for 24 hours. After this time, the reaction mixture was poured into 300 ml water and the mixture was extracted with ethyl acetate. The molecular sieves were removed by filtration and the organic layer was washed several times with dilute HCl and water to remove the pyridine. After drying over magnesium sulfate and evaporation of the solvent in vacuo, the crude mixture was purified by column chromatography (solvent: EtOAc/hexane=1/4). The product was finally recrystallized from methanol to give 0.13 g (yield 6%) as red crystals: mp 134° C. ¹H NMR (500 MHz, 25 ° C., CDCl₃) δ 0.90 (t, J=6.5 Hz, 6 H), 1.33 (m, 12 H), 1.71 (m, 4 H), 1.78 (s, 6 H), 3.46 (t, J=7.75 Hz, 4 H), 6.25 (d, J=3.5 Hz, 1 H), 7.5-8.5 (two broad peaks, 1 H); ¹H NMR (500 MHz, 50° C., CDCl₃) δ 0.91 (t, J=6.75 Hz, 6 H), 1.35 (m, 12 H), 1.72 (m, 4 H), 1.78 (s, 6 H), 3.47 (t, J=7.75 Hz, 4 H), 6.25 (d, J=4.5 Hz, 1 H), 7.91 (s, broad, 1 H); ¹³C NMR (500 MHz, 50° C., CDCl₃) δ 13.85, 22.43, 26.47, 27.10, 27.74, 31.37, 50.57, 54.74, 83.80, 95.45, 108.52, 112.30, 112.94, 113.71, 113.92, 141.18, 164.78, 170.47, 177.33 ppm; IR (neat, cm⁻¹) 2219.

### Example 45

### Preparation of 4-[5-(azepan-2-yl)thien-2-yl]-3-cyano-2-dicyanomethylen-5,5-dimethyl-2,5-dihydrofuran (Entry 18, TH-DCDHF-C6M)

A mixture of 26b (2.0 g, 7.48 mmol), malononitrile (2.5 g, 37.8 mmol), acetic acid (0.02 g) and pyridine (40 ml) was stirred at 90° C. for 24 hours. After this time, the reaction mixture was poured into 300 ml water and the mixture was extracted with ethyl acetate. The organic layer was washed several times with dilute HCl and water to remove the pyridine. After drying over magnesium sulfate and evaporation of the solvent, the crude mixture was purified by column chromatography (solvent: EtOAc/hexane=3/7). The product was finally recrystallized from CH₂Cl₂/methanol to give 0.145 g (yield 5.3%) of red crystals: mp 264° C. ¹H NMR (300 MHz, CDCl₃) δ 1.64 (m, 4 H), 1.77 (s, 6 H), 1.88 (m, 4 H), 3.65 (t, J=5.6 Hz, 4 H), 6.34 (d, J=4.95 Hz, 1 H), 7.5-8.5 (two broad peaks, 1 H); ¹³C NMR (75 MHz, CDCl₃) δ 27.22 (2 carbons), 28.27 (broad), 49.53, 54.12 (broad), 82.76 (broad), 95.76, 108.96, 113.0 (broad), 113.62, 114.14 (broad), 114.55, 141.93, 164.61, 171.44, 177.71 ppm; IR (neat, cm ) 2219.

### Example 46

### Preparation of 1-(3-cyano-2-dicyanomethylen-5,5-dimethyl-2,5-dihydrofuran-4-yl)-2-[5-(N,N-dihexyl)aminothien-2-yl]ethene (Entry 19, TH-DCDHF-6-V)

A mixture of 2-bromo-5-formylthiophene (1 g, 5.23 mmol), dihexylamine (2.9 g, 15.6 mmol) and p-toluenesulfonic acid (0.01 g) was stirred at 120° C. for 24 hours. A mixture of 4-chloroaniline (0.67 g, 5.3 mmol), acetic acid (1.2 g) and ethanol (5 ml) was then added. After stirring at 90-100° C. for 8 more hours, the mixture was cooled and another addition of 21 (0.5 g,2.51 mmol) and pyridine (5 ml) was made. The mixture was kept stirring at room temperature for 8 hours and then poured into 300 ml ice water. The collected precipitate was purified by column chromatography (solvent: EtOAc/hexane/CHCl₃=1/4/5) to give 0.48 g (yield 40%) of product as a purple solid: mp 172° C.; ¹H NMR (300 MHz, CDCl₃) δ 0.91 (t, J=6.6 Hz, 6 H), 1.35 (m, 12 H), 1.67 (s, 6 H), 1.71 (m, 4 H), 3.45 (t, J=7.8 Hz, 4 H), 5.95 (d, br, J=14.4 Hz, 1 H), 6.14 (d, J=4.8 Hz, 1 H), 7.36 (d, J=4.8 Hz, 1 H), 7.75 (app. s, br, 1 H); ¹³C NMR (75 MHz, CDCl₃) δ 14.16, 22.68, 26.67, 27.20, 27.32, 31.58, 49.39, 55.11, 86.24 (br), 95.53, 103.76, 109.50, 113.69 (br), 113.94, 114.64, 125.37, 140.08, 145.09, 170.24, 171.78, 177.21 ppm; IR (neat, cm⁻¹) 2218 (CN), 1595, 1539, 1514.

### Example 47

### Preparation of 3-cyano-2-dicyanomethylen-4-[1-(4-hexylphenyl)-1,4-dihydropyridin-4-yl idenemethylene]-5,5-dimethyl-2,5-dihydrofuran (Entry 28, HP-DDCDHF)

A mixture of 30b (0.28 g, 1.1 mmol), 21 (0.22 g, 1.1 mmol) and acetic anhydride (4 ml) were heated under reflux for 6 hours and then poured into water (50 ml). The precipitate was collected, washed with water, dissolved in ethyl acetate (200 ml), dried over magnesium sulfate, concentrated in vacuo and chromatographed (ethyl acetate/hexane 3/7). Red crystals were obtained (0.13 g, 27%): mp 193° C.; ¹H NMR (300 MHz, CDCl₃) δ 0.88 (t, J=6.9 Hz, 3 H), 1.31 (m, 4 H), 1.54 (s, 6 H), 1.61 (m, 4 H), 2.69 (t, J=7.5 Hz, 2 H), 5.39 (s, 1 H), 7.36 (m, 5 H), 7.93 (m, 3 H); ¹³C NMR (75 MHz, CDCl₃) δ 14.24, 22.72, 27.37, 29.01, 31.34, 31.76, 35.61, 45.96, 82.14, 95.37, 96.64, 115.07, 115.31, 115.98, 122.44, 122.97, 130.75, 138.18, 140.13, 145.95, 152.11, 167.80, 179.96 ppm.

### Example 48

### Preparation of 3-cyano-2-dicyanomethylen-4-[1-(4-perfluorohexylphenyl)-1,4-dihydropyridin-4-ylidenemethylene]-5,5-dimethyl-2,5-dihydrofuran (Entry 27, PFP-DDCDHF)

A mixture of 30a (0.66 g, 1.35 mmol), 21 (0.25 g, 1.25 mmol) and acetic anhydride (5 ml) was heated under reflux for 24 hours and then poured into water (100 ml). The precipitate was collected, washed with water, dissolved in ethyl acetate, dried over magnesium sulfate, concentrated in vacuo and chromatographed (ethyl acetate/hexane 3/7). A deep cherry glass was obtained (0.47 g, 56%): glass 157° C. crystal 184° C. I; ¹H NMR (300 MHz, CDCl₃) δ 1.57 (s, 6 H), 5.40 (s, 1 H), 7.27 (d, J=6.6 Hz, 2 H), 7.67 (d, J=8.4 Hz, 2 H), 7.85 (d, J=8.4 Hz, 2 H), 7.92 (d, J=6.6 Hz, 2 H); ¹³C NMR (75 MHz, CDCl₃) δ 27.18, 47.14, 79.68, 95.90, 97.08, 114.69, 114.95, 115.43, 122.06, 123.53, 129.73, 130.64 (t, J=24.9 Hz), 137.34, 145.05, 151.88, 169.30, 179.82, carbons in the perfluoroalkyl chain cannot be identified because of low intensities and coupling with fluorine; ¹⁹F NMR (282 MHz, CDCl₃) δ −81.18 (m, 3 F), −111.30 (m, 2 F), −121.75 (m, 2 F), −121.96 (m, 2 F), 123.19 (m, 2 F), 126.53 (m, 2 F).

### Example 49

### Preparation of 3-cyano-2-dicyanomethylen-4-[1-(4-dodecyloxycarbonylphenyl)-1,4-dihydropyridin-4-ylidenemethylene]-5,5-dimethyl-2,5-dihydrofuran (Entry 29, DOCP-DDCDHF)

A mixture of 30c (2.3 g, 6.0 mmol), 21 (1 g, 5 mmol) and acetic anhydride (20 ml) was heated under reflux for 24 hours and then poured into water (500 ml). The precipitate was collected, washed with water, dissolved in ethyl acetate, dried over magnesium sulfate, concentrated and chromatographed (ethyl acetate/hexane 3/7). A black solid was obtained (0.85 g, 30%): a glass, no melting point observed; ¹H NMR (300 MHz, CDCl₃) δ 0.87 (t, J=6.6 Hz, 3 H), 1.25-1.82 (m, 20 H), 1.59 (s, 6 H), 4.35 (t, J=6.6 Hz, 2 H), 5.39 (s, 1 H), 7.31 (app. s, br, 2 H), 7.56 (app. s, br, 2 H), 7.92 (app. s, br, 2 H), 8.26 (app. s, br, 2 H); ¹³C NMR (75 MHz, CDCl₃) δ 14.29, 22.84, 26.14, 27.27, 28.80, 29.42, 29.49, 29.68, 29.74, 29.78 (2 carbons), 32.06, 47.37, 66.15, 79.76, 95.80, 96.86, 114.64, 114.82, 115.40, 122.06, 123.01, 132.10, 132.22, 137.35, 145.32, 151.78, 165.03, 169.26, 179.77.

### Example 50

### Preparation of 1-(4-hydroxyphenyl)-2,6-dimethyl-1H-pyridin-4-one hydrochloride 33a

A mixture of dehydroacetic acid (25.4 g, 0.151 mol), 4-hydroxyaniline (15 g, 0.137 mol) and conc. HCl (32 ml) was stirred in a 300 ml round bottom flask fitted with a rotary evaporator trap, a stir bar and a bubbler. The mixture was gradually warmed in an oil bath. At 130° C., a clear solution was obtained and gas evolution occurred. The bath temperature was slowly raised to 160° C. and kept at this temperature for 2 hours until gas evolution ceased. The mixture was cooled down to room temperature and a large amount of white crystals precipitated. Acetone was added to help with crystallization. Crystals were then collected by suction filtration and washed with acetone. The white crystals obtained (26.6 g, 77%) were used directly for the next step: m.p.>300° C.

### Example 51

### Preparation of 1-(3-hydroxyphenyl)-2,6-dimethyl-1H-pyridin-4-one hydrochloride 33b

A mixture of dehydroacetic acid (25.4 g, 0.151 mol), 3-hydroxyaniline (15 g, 0.137 mol) and concentrated HCl (32 ml) was stirred in a 300 ml round bottom flask fitted with a rotary evaporator trap, a stir bar and a bubbler. The mixture was gradually warmed in an oil bath. At 130° C., a clear solution was obtained and gas evolution occurred. The bath temperature was slowly raised to 160° C. and kept at this temperature for 2 hours until gas evolution ceased. The mixture was cooled down to room temperature and a large amount of white crystals precipitated. Acetone was added to help with crystallization. Crystals were then collected by suction filtration and washed with acetone. The obtained white crystals (24.5 g, 71%) were used directly for the next step: m.p. 288° C.

### Example 52

### Preparation of 1-[4-(2-ethyl hexyloxy)phenyl]-2,6-dimethyl-1H-pyridin-4-one 34a

A mixture of potassium carbonate (11 g, 80 mmol), 33a (5 g, 20 mmol), 2-ethylhexyl bromide (4.6 g, 24 mmol), NMP (40 ml) and traces of potassium iodide was stirred at 110° C. for 4 hours. The mixture was then poured into water (500 ml). The precipitate was collected by suction filtration and recrystallized from ethyl acetate/hexane to give white crystals (2.64 g, 41%); mp 143° C.; ¹H NMR (300 MHz, CDCl₃) δ 0.86 (t, J=7.2 Hz, 3 H), 0.89 (t, J=7.5 Hz, 3 H), 1.3-1.5 (m, 8 H), 1.70 (m, 1 H), 1.86 (s, 6 H), 3.84 (d, J=5.7 Hz, 2 H), 6.20 (s, 2 H), 6.95 (d, J=9.0 Hz, 2 H), 7.04 (d, J=9.0 Hz, 2 H); ¹³C NMR (75 MHz, CDCl₃) δ 11.26, 14.21, 21.64, 23.13, 23.93, 29.19, 30.59, 39.46, 71.00, 115.70, 117.43, 128.89, 131.95, 149.51, 160.00, 179.57 ppm.

### Example 53

### Preparation of 1-[3-(2-ethylhexyloxy)phenyl]-2,6-dimethyl-1H-pyridin-4-one 34b

A mixture of potassium carbonate (22 g, 159 mmol), 33b (10 g, 40 mmol), 2-ethylhexyl bromide (9.2 g, 48 mmol), NMP (100 ml) and traces of potassium iodide was stirred at 110° C. for 4 hours. The mixture was then poured into water and extracted with ethyl acetate. The organic layer was washed several times with water to get rid of NMP, dried over magnesium sulfate, concentrated in vacuo and chromatographed over silica gel (ethyl acetate:methanol=4:1): clear glass (6.5 g, 50% yield); ¹H NMR (300 MHz, CDCl₃) δ 0.84 (t, J=6.9 Hz, 3 H), 0.88 (t, J=7.5 Hz, 3 H), 1.17-1.46 (m, 8 H), 1.68 (m, 1 H), 1.90 (s, 6 H), 3.80 (d, J=5.7 Hz, 2 H), 6.21 (s, 2 H), 6.66 (dd, J=2.4, 1.8 Hz, 1 H), 6.70 (ddd, J=7.8, 1.8, 0.9 Hz, 1 H), 6.98 (ddd, J=8.4, 2.4, 0.9 Hz, 1 H), 7.37 (dd, J=8.4, 7.8 Hz, 1 H); ¹³C NMR (75 MHz, CDCl₃) δ 11.23, 14.18, 21.37, 23.10, 23.90, 29.17, 30.55, 39.44, 71.20, 114.30, 115.78, 117.37, 119.76, 130.89, 140.50, 148.93, 160.76, 179.56 ppm.

### Example 54

### Preparation of 3-cyano-2-dicyanomethylen-4-{1-[4-(2-ethylhexyloxy)phenyl]-2,6-dimethyl-1,4-dihydropyridin-4-ylidenemethylene}-5,5-dimethyl-2,5-dihydrofuran (Entry 31, 2EHO-DDCDHF)

A mixture of 34a (2.5 g, 7.6 mmol), 21 (1.52, 7.6 mmol) and acetic anhydride (15 ml) was refluxed for 6 hours and then poured into water (400 ml). The precipitate was collected, washed with water, dissolved in ethyl acetate, dried over magnesium sulfate, concentrated and chromatographed (ethyl acetate/hexane 3/7). After recrystallization from dichloromethane/methanol, red crystals were obtained (0.55 g, 14%): mp 237° C.; ¹H NMR (300 MHz, CDCl₃) δ 0.88 (t, J=6.9 Hz, 3 H), 0.92 (t, J=7.5 Hz, 3 H), 1.30-1.50 (m, 8 H), 1.49 (s, 6 H), 1.74 (m, 1 H), 2.19 (s, 6 H), 3.88 (d, J=5.7 Hz, 2 H), 5.27 (s, 1 H), 7.06 (d, J=9.0 Hz, 2 H), 7.12 (d, J=9.0 Hz, 2 H), 7.19 (s, 2 H); ¹³C NMR (75 MHz, CDCl₃) δ 11.28, 14.24, 22.14, 23.15, 23.93, 27.65, 29.20, 30.58, 39.43, 43.65, 71.21, 74.91, 94.63, 96.45, 116.00, 116.07, 116.44, 116.86, 122.08, 127.48, 130.74, 150.84, 153.07, 160.88, 165.23, 179.93 ppm.

### Example 55

### Preparation of 3-cyano-2-dicyanomethylen-4-{1-[3-(2-ethylhexyloxy)phenyl]-2,6-dimethyl-1,4-dihydropyridin-4-ylidenemethylene}-5,5-dimethyl-2,5-dihydrofuran (Entry 32, M2EHO-DDCDHF)

A mixture of 34b (4.8 g, 14.7 mmol), 21 (2.92 g, 14.7 mmol) and acetic anhydride (50 ml) was heated under reflux for 8 hours and then poured into water (500 ml). The precipitate was collected, washed with water, dissolved in ethyl acetate, dried over magnesium sulfate, concentrated in vacuo and chromatographed (dichloromethane:ethyl acetate=20:1). After recrystallization from CH₂Cl₂/methanol, red crystals were obtained (1.36 g, 18% yield): mp 181° C.; ¹H NMR (300 MHz, CDCl₃) δ 0.88 (t, J=7.2 Hz, 3 H), 0.92 (t, J=7.2 Hz, 3 H), 1.24-1.57 (m, 8 H), 1.51 (s, 6 H), 1.73 (m, 1 H), 2.24 (s, 6 H), 3.85 (d, J=5.7 Hz, 2 H), 5.27 (s, 1 H), 6.74 (dd, J=2.4, 1.8 Hz, 1 H), 6.77 (ddd, J=7.8, 1.8, 0.6 Hz, 1 H), 7.10 (ddd, J=8.4, 2.4, 0.6 Hz, 1 H), 7.18 (s, 2 H), 7.50 (dd, J=8.4, 7.8 Hz, 1 H); ¹³C NMR (75 MHz, CDCl₃) δ 11.26, 14.22, 21.88, 23.13, 23.90, 27.63, 29.18, 30.54, 39.43, 43.70, 71.50, 75.19, 94.67, 96.19, 112.64, 115.90, 115.95, 116.71, 117.02, 118.00, 121.90, 131.80, 139.37, 150.06, 153.07, 161.30, 165.65, 179.99 ppm.

### Example 56

### Preparation of 3-cyano-2-dicyanomethylen-4-(1-phenyl-2,6-dimethyl-1,4-dihydropyridin-4-ylidenemethylene)-5,5-dimethyl-2,5-dihydrofuran (Entry 30, P-DDCDHF)

Using the same method just described for 2EHO-DDCDHF and M2EHO-DDCDHF, a mixture of 1-phenyl-2,6-dimethyl-1H-pyridin-4-one hydrogen chloride 33c (0.65 g, 2.76 mmol), 21 (0.5 g, 2.51 mmol) and acetic anhydride (10 ml) was reacted to give red crystals (120 mg, yield 13%) as the product: mp 290° C. ¹H NMR (300 MHz, CDCl₃) δ 1.53 (s, 6 H), 2.18 (s, 6 H), 5.23 (s, 1 H), 7.15 (s, 2 H), 7.25 (m, 1 H), 7.65 (m, 4 H).

### Example 57

### Preparation of 3-cyano-2-dicyanomethylen-4-[4-(4-diethylaminophenyl)buta-1,3-dienyl]-5,5-dimethyl-2,5-dihydrofuran (entry 33, DCDHF-2-2V)

Under the protection of nitrogen, a mixture of aldehyde 35 (1.01 g, 5 mmol), 4-chloroaniline (1.27 g, 10 mmol), acetic acid (1.2 g, 19.9 mmol) and ethanol (20 ml) was stirred at room temperature. A red mixture was obtained immediately. Two hours later, the starting aldehyde disappeared and two new red spots showed up from TLC. The reaction mixture was further stirred at room temperature for two more hours. Without any workup, the reaction mixture was cooled to 0° C. in an ice bath. A mixture of 21 (0.99 g, 5 mmol) and pyridine was then added. After two more hours at this temperature, TLC showed that the reaction was finished. The reaction mixture was poured into 600 ml ice water in a beaker. The precipitate was collected and washed with water. The solid obtained was dissolved in chloroform, dried over MgSO₄, filtered through a short pad of silica gel and concentrated. The obtained crude product was crystallized from CHCl₃/EtOH. Black crystals (1.54 g, 81% yield) were obtained as the product: no melting point observed before decomposition temperature of 239° C. ¹H NMR (300 MHz, CDCl₃) δ 1.23 (t, J=7.1 Hz, 6 H), 1.69 (s, 6 H), 3.45 (q, J=7.1 Hz, 4 H), 6.33 (d, J=15.0 Hz, 1 H), 6.67 (d, J=9.0 Hz, 2 H), 6.83 (dd, J=14.7, 15.0 Hz, 1 H), 7.14 (d, J=14.7 Hz, 1 H), 7.43 (d, J=9.0 Hz, 2 H), 7.63 (dd, J=14.7, 15.0 Hz, 1 H); ¹³C NMR (75 MHz, CDCl₃) δ 12.83, 26.79, 44.97, 54.56, 93.91, 96.96, 111.83, 111.95, 112.20, 112.98, 114.26, 122.46, 122.86, 131.64, 149.55, 150.58, 150.75, 173.75, 176.50.

### Example 58

### Photophysical Properties of Fluorophore Compounds

The chemical structures from FIGS. 1-11 were evaluated for their λmax (in THF), melting point Mp (by differential scanning calorimetry or “DSC”), Tg, Trec, Td (by thermogravimetric analysis or “TGA”), and position of highest occupied molecular orbital (or “HOMO”). Tg (glass transition temperature) and Trec (recrystallization temperature) were measured by cooling melted samples (a cooling rate of 10° C. per minute was generally used. A rate of 30° C. per minute was used for samples indicated by a*, and 5° C. per minute for samples indicated with a #, followed by second heating at 10° C. per minute. Two recrystallization temperatures were recorded: first number is the onset value of the recrystallization and second number is the peak value. TGA is measured by heating the sample from room temperature to 1000° C. Td is the decomposition temperature determined from both TGA and DSC. HOMO is calculated from a cyclic voltammetry (“CV”) measurement. Conditions for CV: Pt electrode, Pt disk and Hg/HgCl₂/NaCl reference electrode, 0.1 M tetraethylammonium tetrafluroborate in acetonitrile as supporting electrolyte, speed: 300 mV per second.

| | λmax(THF) | | | | | |
|---|---|---|---|---|---|---|
| | (␣ₘₐₓ | | | | | |
| Compound | (L cm¹ mol¹)) | Mp (° C.) | Tg (° C.) | T_{rec} (° C.) | Td (° C.) | HOMO (eV) |
| 1 | 486 | 183 | 36^{#} | 71^{#} | 312 | −5.63 |
| | (68600) | | | 89^{#} | | |
| 2 | 483 | >278 | no | no | 278 | Insol |
| | | | | | (at mp) | |
| 3 | 491 | 249 | 16* | 122* | 299 | |
| | (74300) | | | 134* | | |
| 4 | 491 | 305 | no | no | 319 | |
| | (62800) | | | | | |
| 5 | 488 | 250 | 69 | 107 | 292 | |
| | (64400) | | | 131 | | |
| 6 | 490 | 278 | no | no | 313 | |
| | (76800) | | | | | |
| 7 | 491 | 250 | no | no | 311 | −5.59 |
| | (72600) | | | | | |
| 8 | 491 | 169 | no | no | 312 | |
| | (77000) | | | | | |
| 9 | 491 | 129 | 19^{#} | 75^{#} | 320 | −5.56 |
| | (72500) | | | 91^{#} | | |
| 10 | 492 | 123 | 1^{#} | 66 | 322 | |
| | (76700) | | | 78^{#} | | |
| 11 | 492 | 171 | 12 | 58 | 313 | −5.57 |
| | (70500) | | | 64 | | |
| 12 | 492 | 150 | 33 | 84 | 318 | −5.54 |
| | (74700) | | | 99 | | |
| 13 | 493 | 95 | 2^{#} | Stable^{#} | 326 | |
| | (74300) | | | | | |
| 14 | 520 | 214 | 76^{#} | stable^{#} | 275 | |
| | (75400) | | | | | |
| 15 | 520 | 130 | 17^{#} | Stable^{#} | 310 | −5.61 |
| | (75800) | | | | | |
| 16 | 517 | 180 | 64^{#} | Stable^{#} | 277 | |
| | (75800) | | | | | |
| 17 | 515 | 134 | 22^{#} | 98^{#} | 308 | −5.47 |
| | (117500) | | | 113^{#} | | |
| 18 | 513 | 264 | 89^{#} | 146^{#} | 332 | −5.46 |
| | (118000) | | | 170^{#} | | |
| 19 | 620 | 172 | no | no | 298 | −5.16 |
| | (172000) | | | | | |
| 20 | 570 | 245 | no | no | 246 | −5.32 |
| | (70200) | | | | (at mp) | |
| 21 | 606 | 243 | no | no | 239 | |
| | (69100) | | | | (at mp) | |
| 22 | 577 | 140K147 | 34 | 107 | 262 | |
| | (76600) | | | | | |
| 23 | 574 | 100 | 22 | Stable | 309 | −5.32 |
| | (50900) | | | | | |
| 24 | 561 | 212 | no | no | 278 | −5.34 |
| | (65500) | | | | | |
| 25 | 537 | 331 | no | no | 336 | −5.45 |
| | (50000) | | | | (at mp) | |
| 26 | 572 | glass | 141 | Stable | 270 | |
| | (67200) | | | glass | | |
| 27 | 540 | Glass157 | 103 | 154 | 309 | −5.32 |
| | (83000) | crystal184 | | 168 | | Irreversible |
| | | | | | | onset value |
| 28 | 531 | 193 | 57 | Stable | 318 | −5.28 |
| | (87600) | | | glass | | Irreversible |
| | | | | | | onset value |
| 29 | 540 | glass | 64 | Stable | 314 | −5.29 |
| | (85800) | | | glass | | Irreversible |
| | | | | | | onset value |
| 30 | | 290 | no | no | 310 | |
| 31 | 511 | 237.4 | 76 | 112 | 320 | −5.24 |
| | (99000) | | | 118 | | Irreversible |
| | | | | | | onset value |
| 32 | 511 | 181 | 69 | Stable | 324 | −5.23 |
| | (98000) | | | glass | | Irreversible |
| | | | | | | onset value |
| 33 | 606 | No mp | no | no | 239 | |
| | (66200) | | | | | |

### Example 59

### Design of Calcium Binding Fluorophores

Commercially available calcium detecting compounds such as R-1244 (Molecular Probes, Eugene, Oreg.) contains a conventional calcium Ca²⁺ chelating ligand covalently attached to a conventional fluorescent oxazine dye.

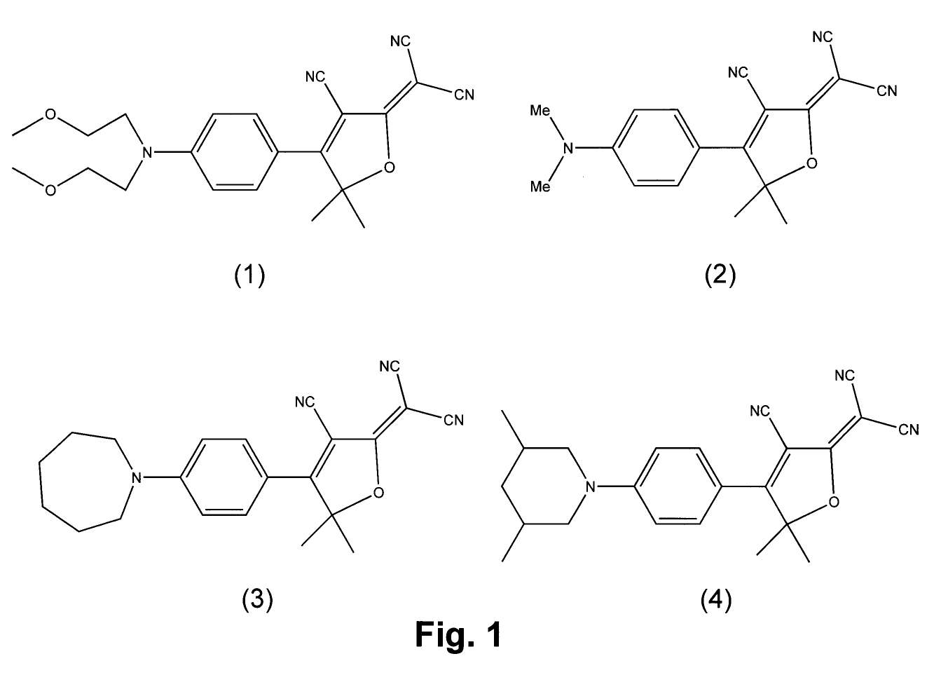

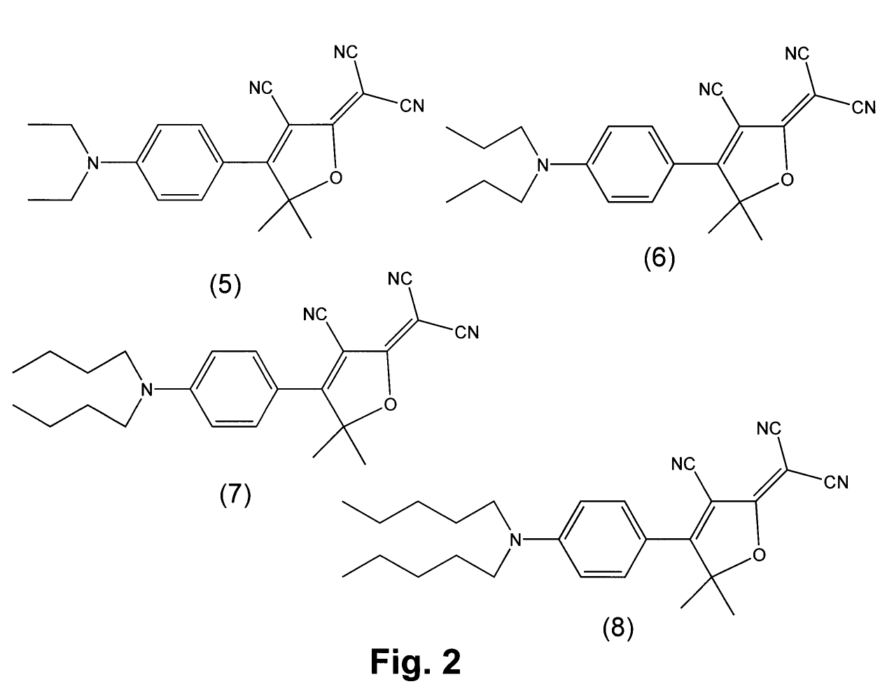

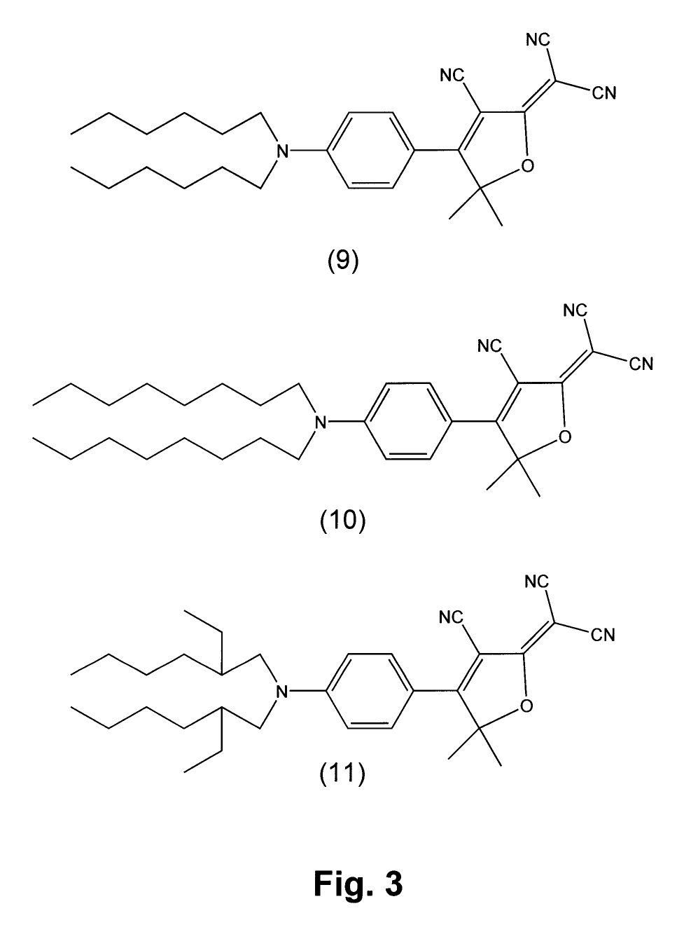

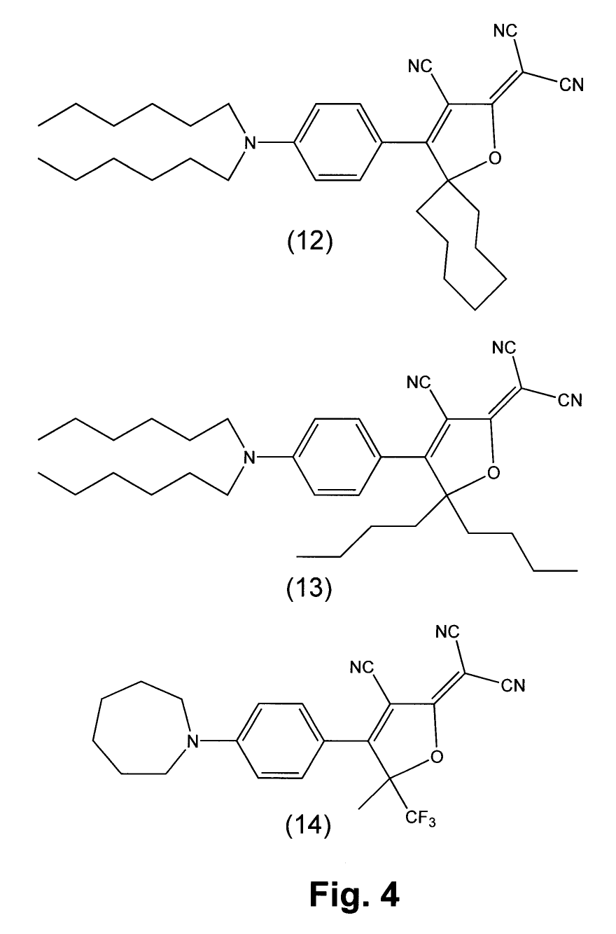

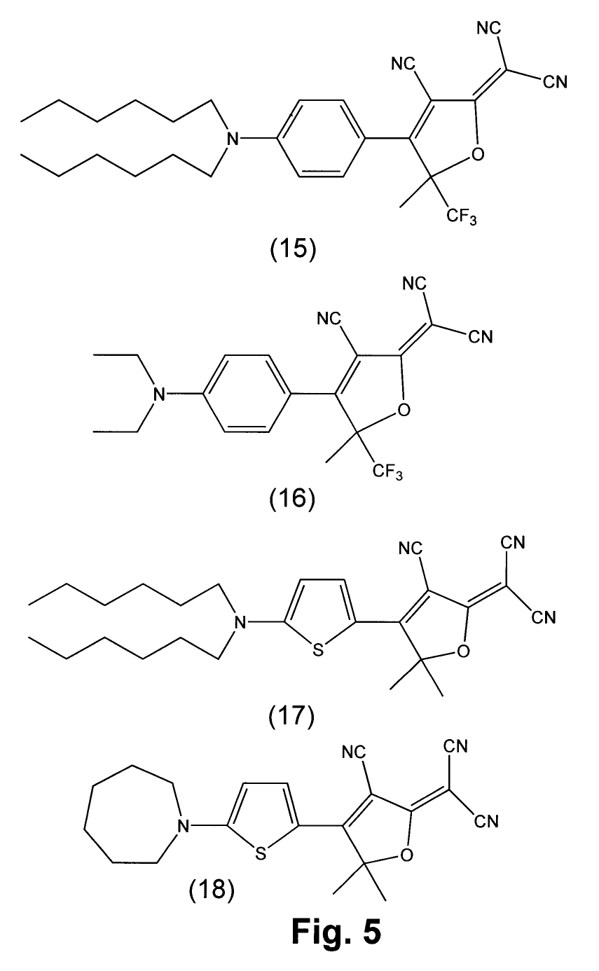

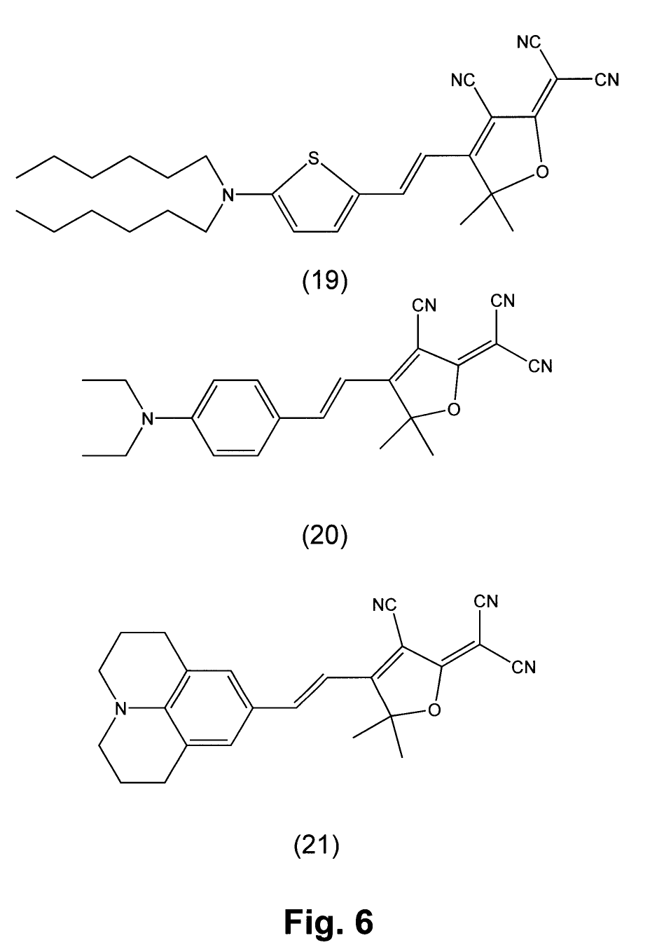

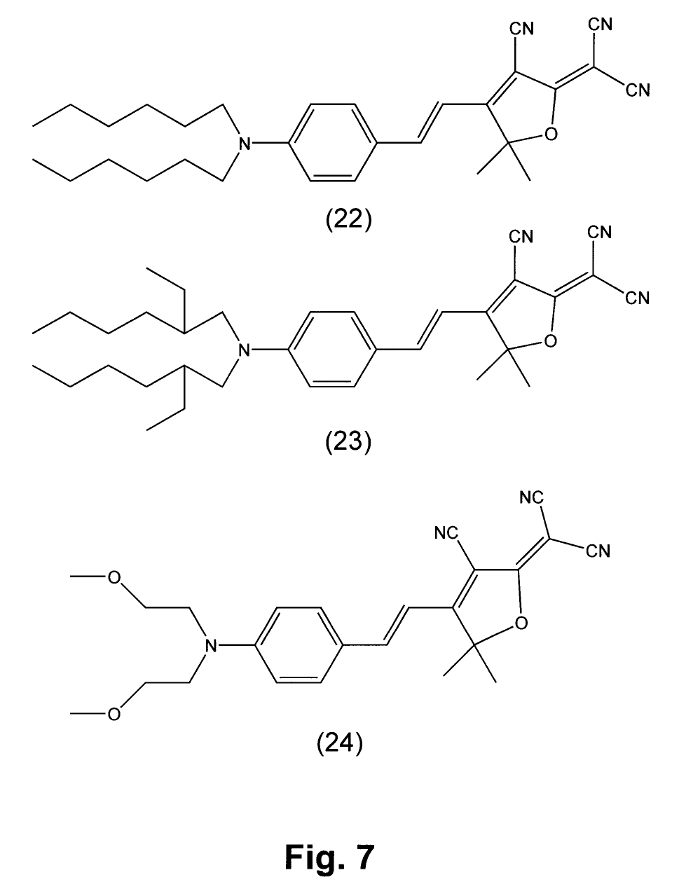

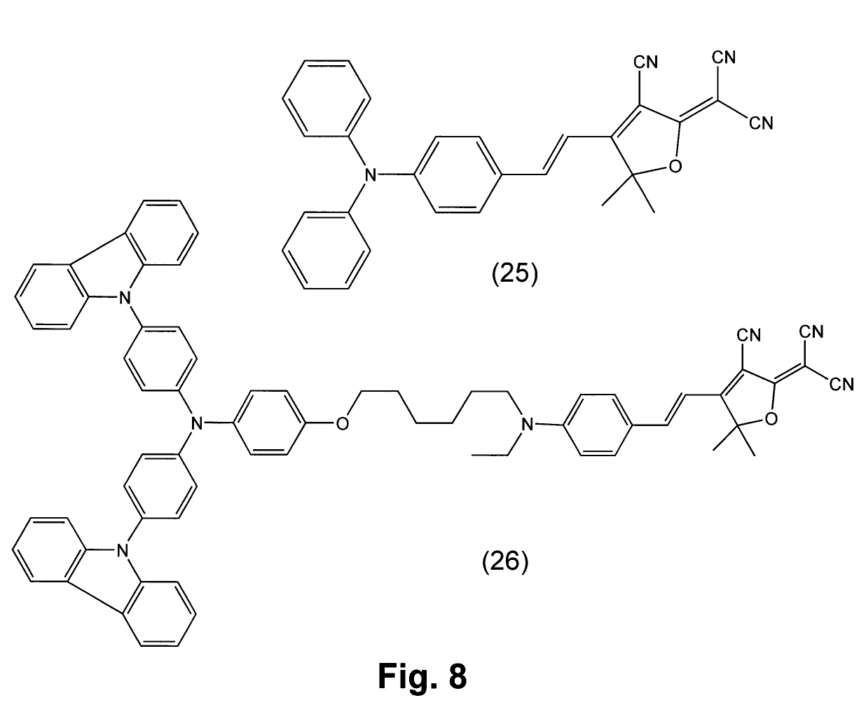

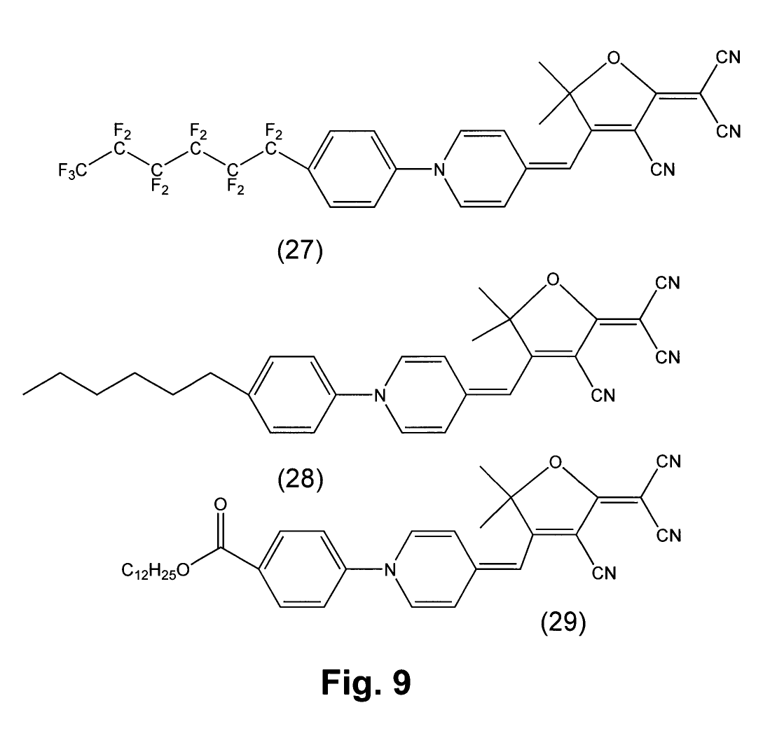

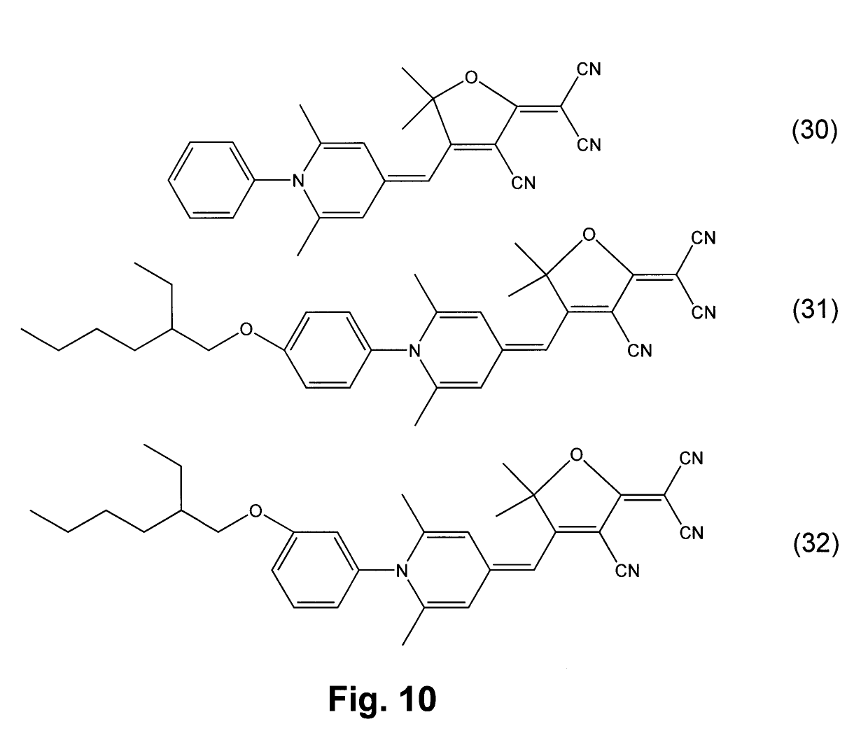

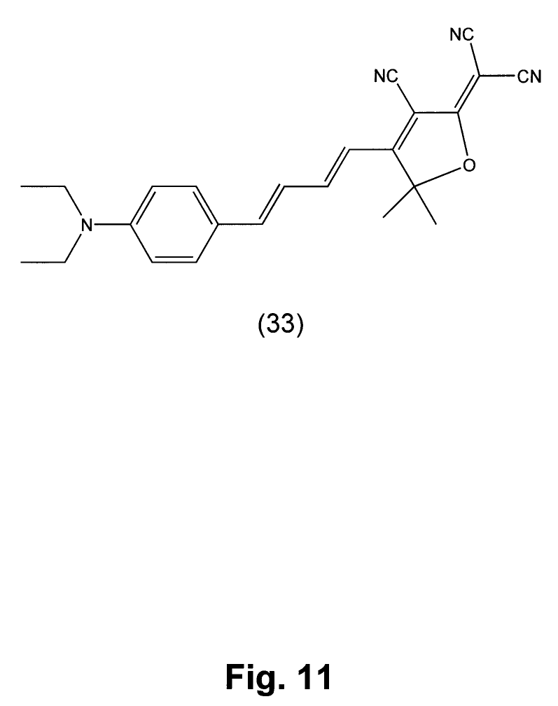

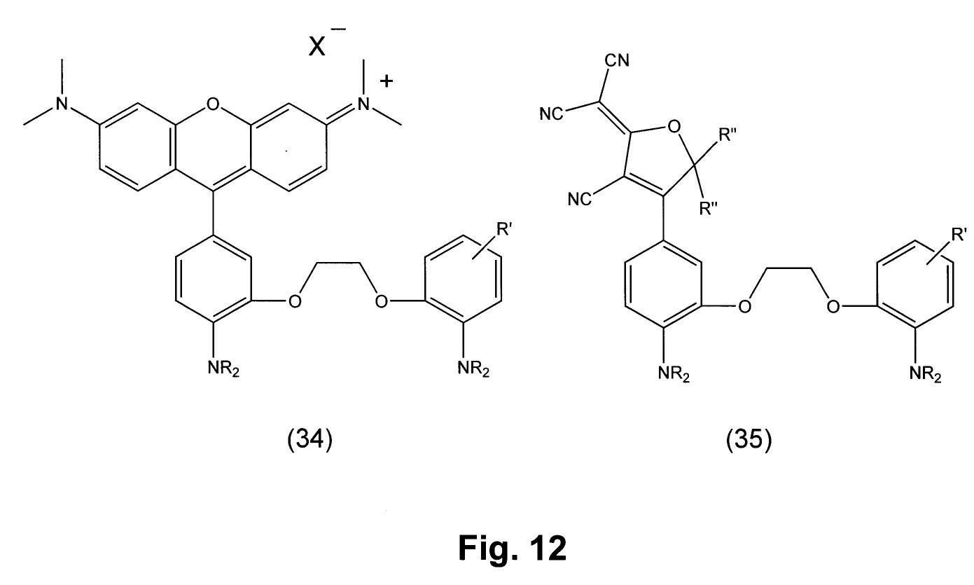

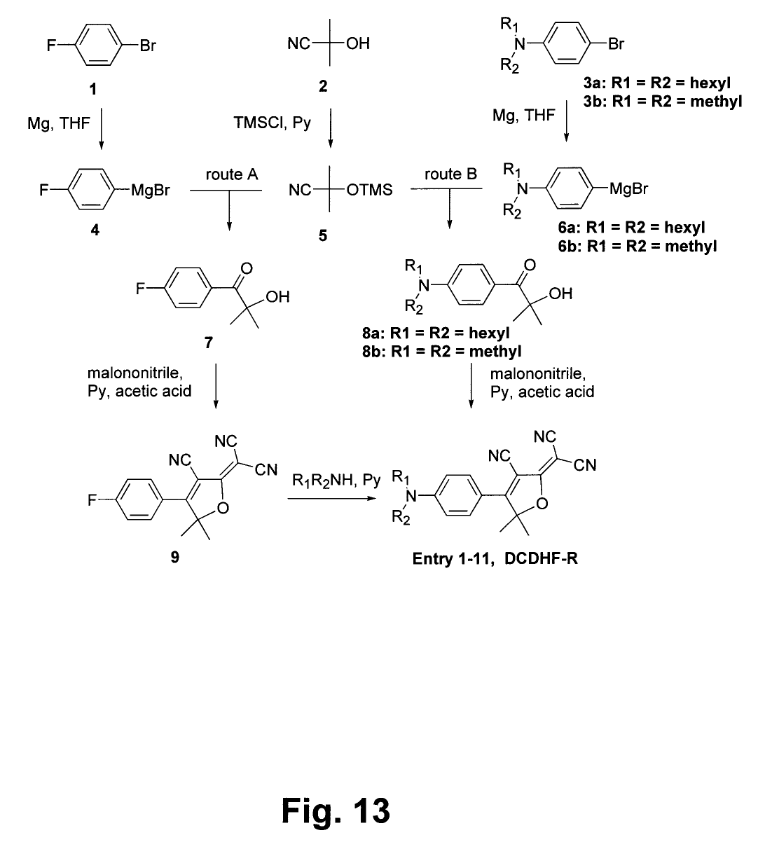

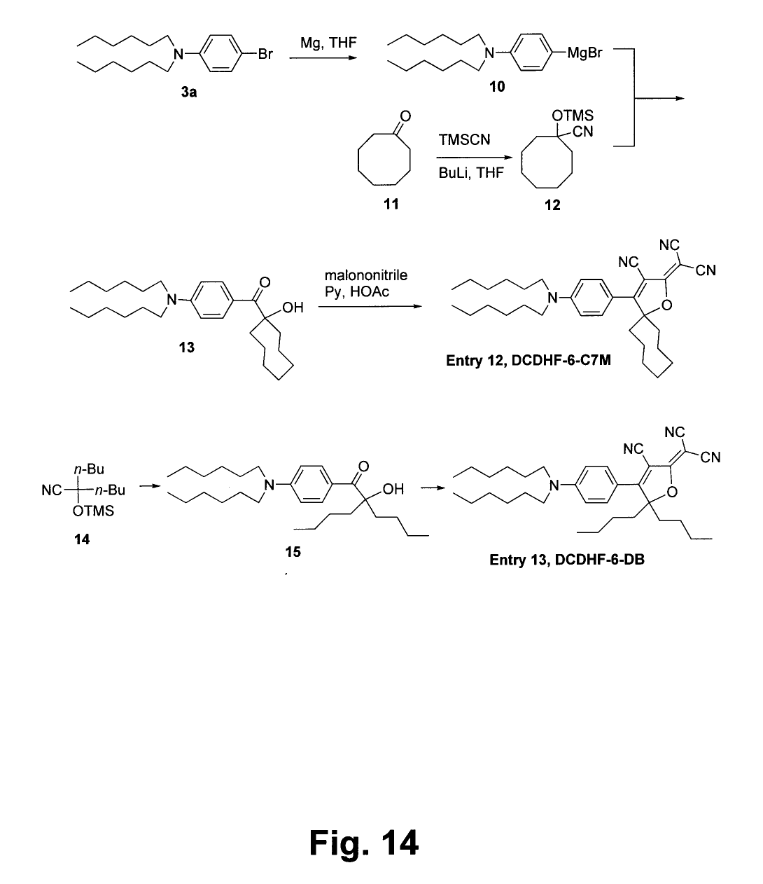

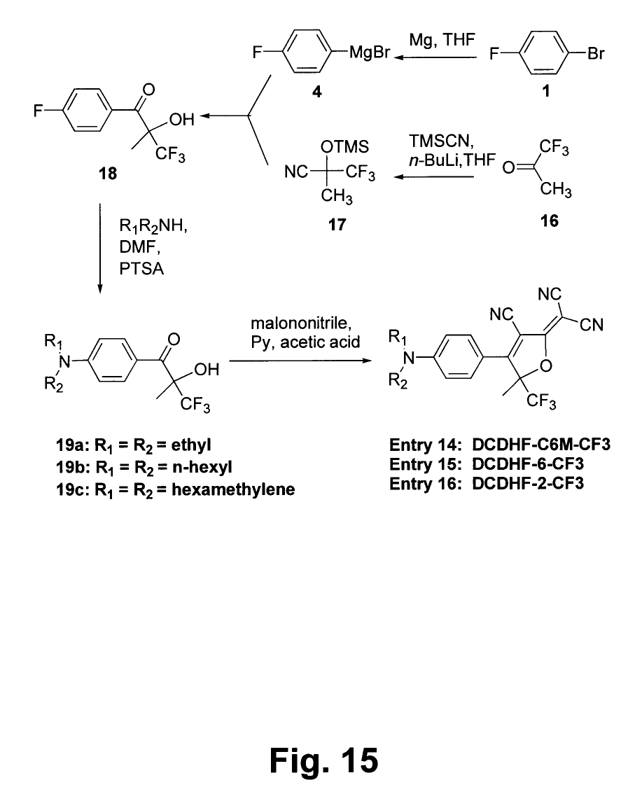

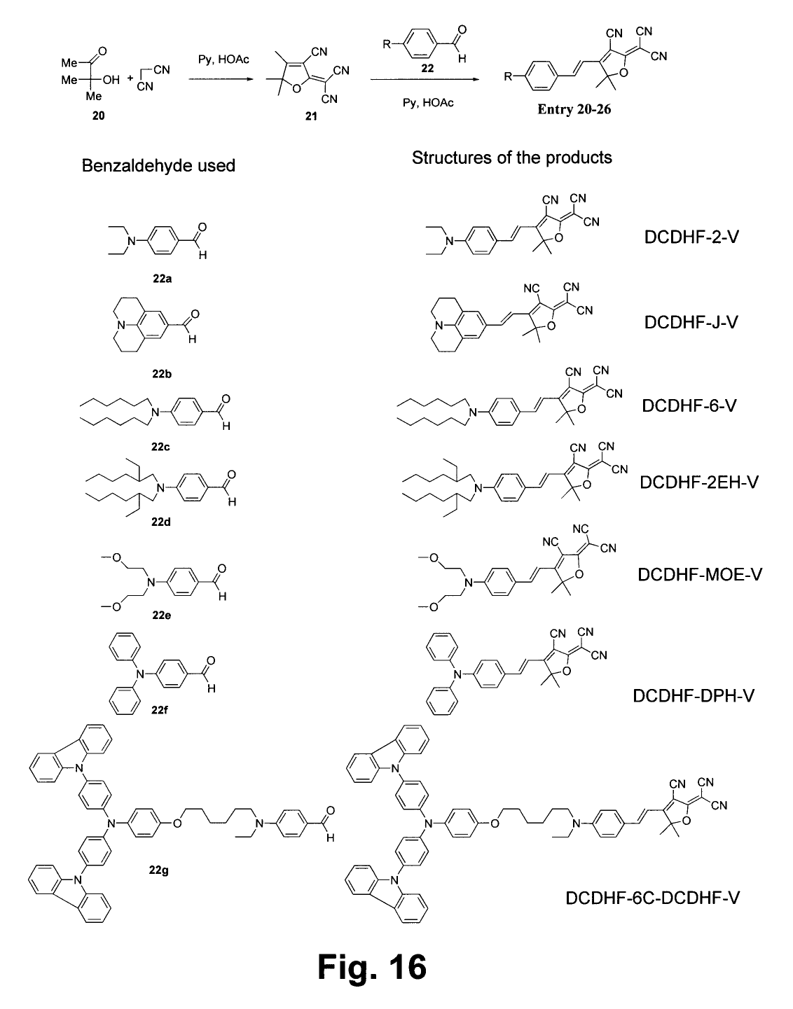

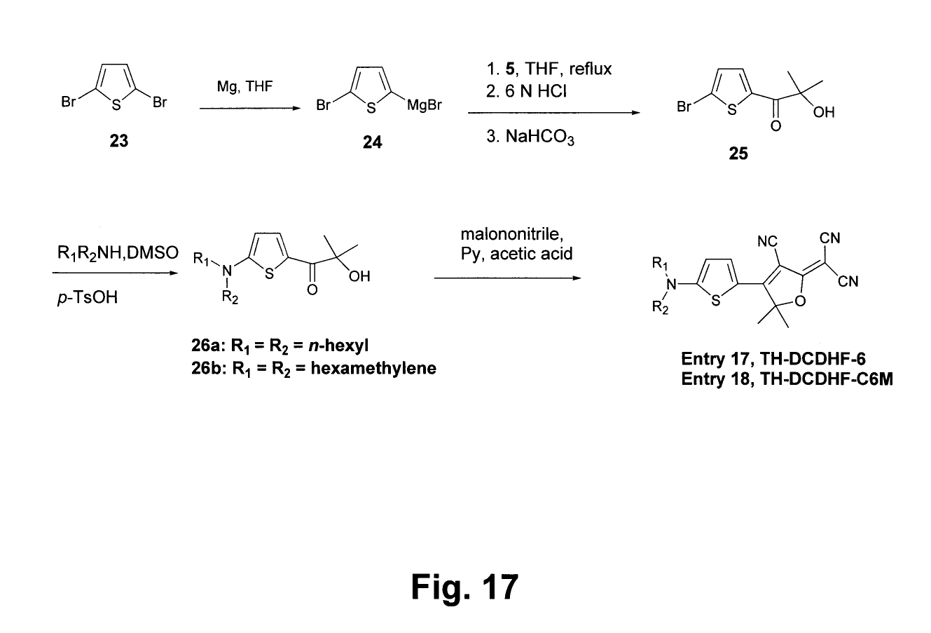

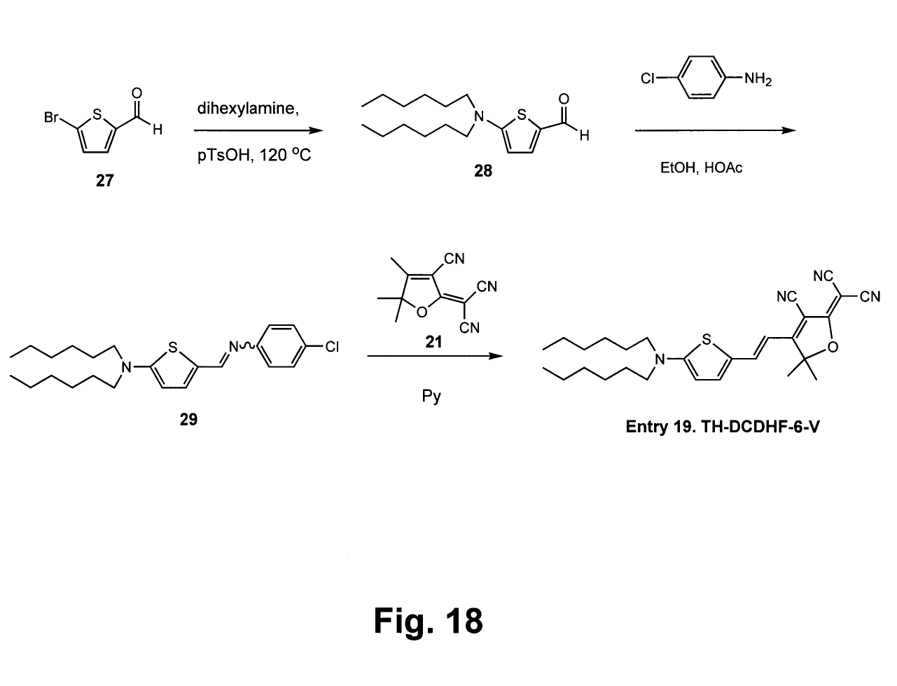

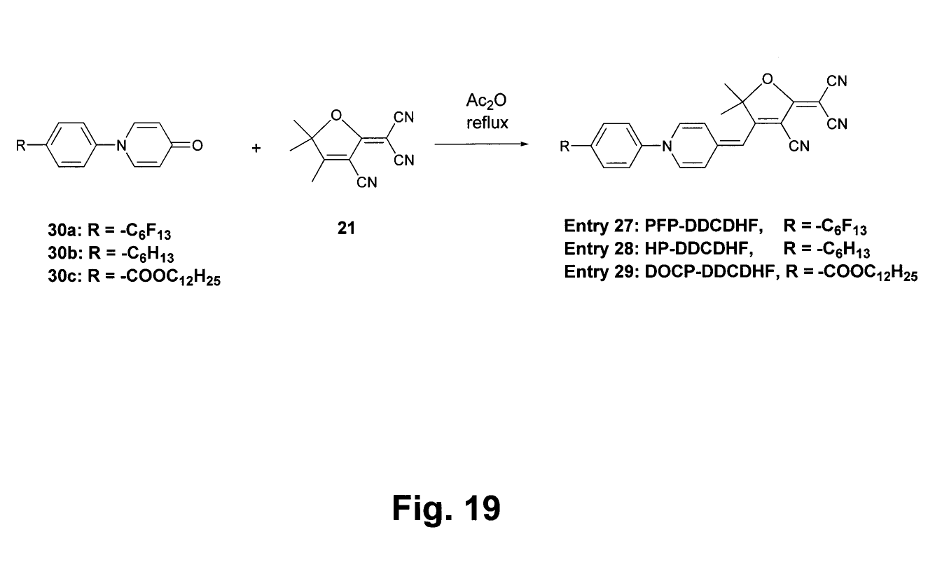

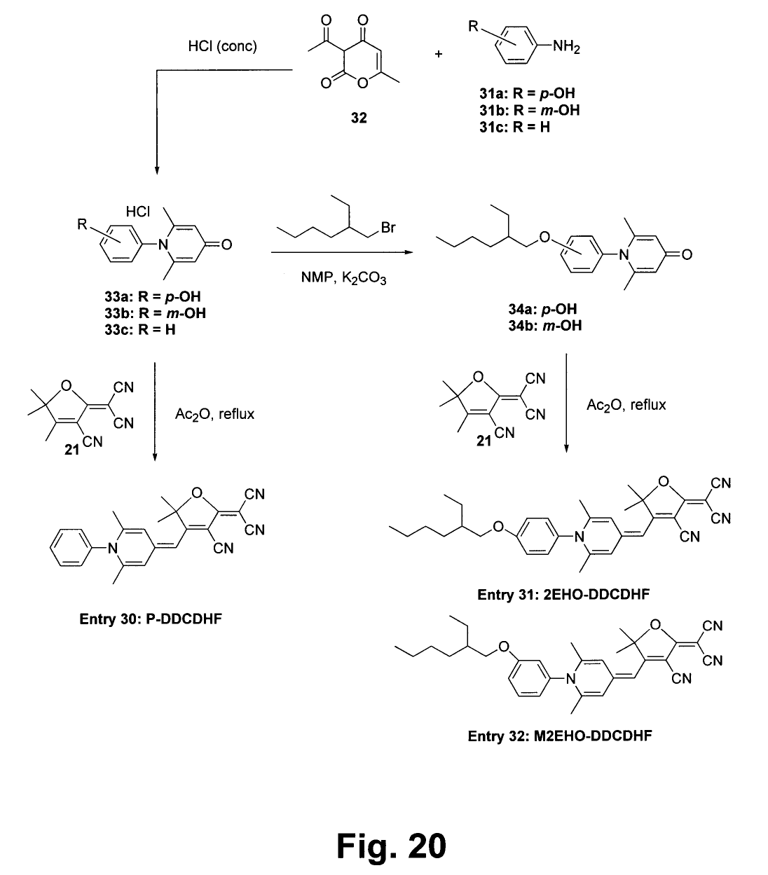

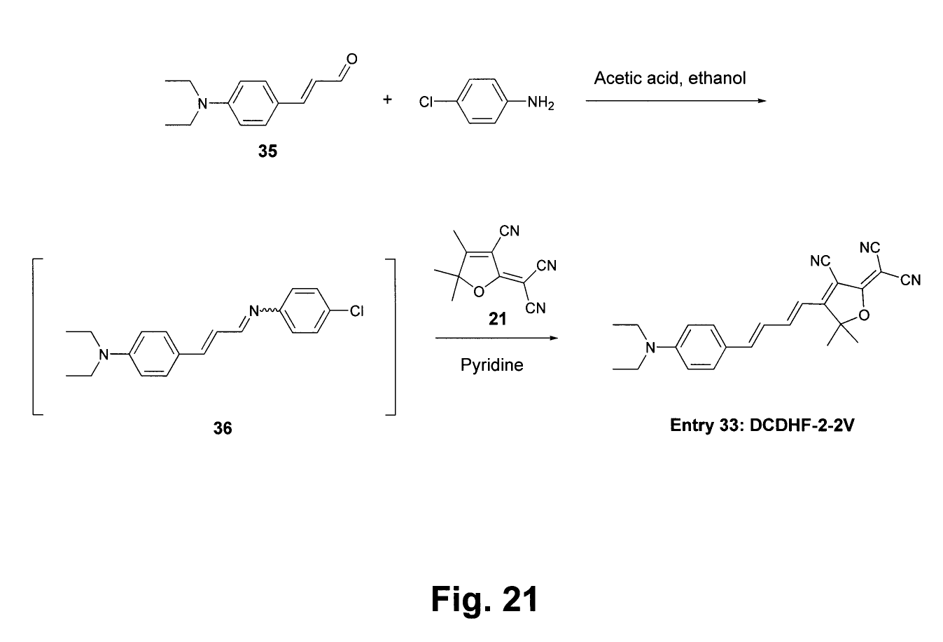

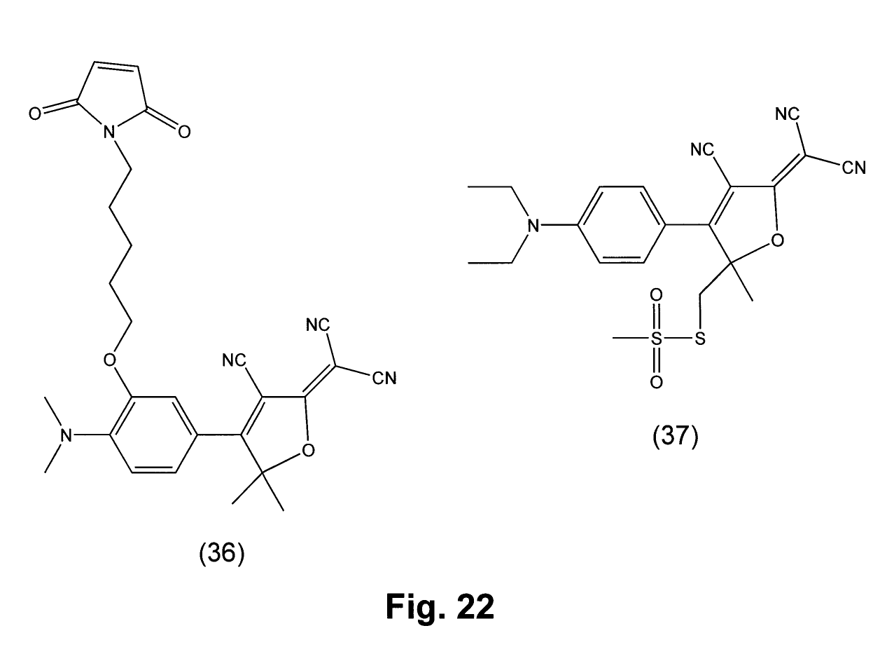

Metal ion ligands can be covalently attached to a DCDHF fluorophore to afford novel metal ion detecting compounds. FIG. 12 shows the structure of R-1244 and a class of metal ligand DCDHF fluorophores. The fluorophore properties could be modulated by altering the R, R′, and R″ groups. The fluorescence may be more sensitive to the calcium binding than in R-1244. A second DCDHF fluorophore could be incorporated as R′, to give a symmetrical molecule. This structure would be expected to have significant conformational mobility in the absence of calcium ions, but would be significantly less free when bound to a calcium ion. As fluorescence properties are highly sensitive to chromophore density and alignment, an enhanced response to the binding event is expected.

The development of such metal ligand-DCDHF compounds is expected to facilitate measurement of intracellular free calcium concentrations during calcium signaling in electrically excitable and non-excitable cells. For example, imaging of calcium transients in mammalian eggs would be possible. The activation of eggs at fertilization depends on the generation of repeated, transient calcium waves. The metal ligand-DCDHF compounds could be used to measure calcium in the endoplasmic reticulum, cytoplasm, and in mitochondria. This is but one example of how these compounds could be used to measure metal ion concentrations in biological systems.

### Example 60

### In Vivo Labeling of Cells with Fluorophore Compounds

Living Chinese hamster ovary cells (CHO cells) in a standard growth medium were contacted with a solution of compound TH-DCDHF-6V (compound 22; FIG. 7) in ethanol. The treated cells were then washed with buffer. The cells were imaged in buffer using an inverted epifluorescence microscope with 633 nm optical excitation. Regions within the cells were observed to be differentially labeled. These results confirm passage of the fluorophore compound through the cell membrane, and differential binding of the compound to various structures or regions within the cells. Further attachment of long alkyl chains (C10, C12, C14, C16, C18, C20, or C22) to the R¹ and R² positions would likely provide improved degrees of retention in the cellular membranes.

### Example 61

### Labeling of Proteins and Peptides

A fluorophore compound containing a maleimide, iodoacetamide, or methylthiosulfonate group can be contacted with a protein or peptide containing at least one cysteine residue. After a sufficient time for formation of a covalent bond or a disulfide bond, the fluorophore labeled protein or peptide can be purified from unreacted material. The fluorophore compound may have several such functional groups, resulting in attachment to several cysteine residues.

### Example 62

### Labeling of Proteins and Peptides

A fluorophore compound containing an N-hydroxysuccinimide group can be contacted with a protein or peptide containing at least one lysine, asparagine, glutamine, arginine, or histidine residue. After a sufficient time for formation of a covalent bond, the fluorophore labeled protein or peptide can be purified from unreacted material. The fluorophore compound may have several such reactive functional groups, resulting in attachment to several amine-containing amino acid residues.

### Example 63

### Labeling of Nucleic Acids

A fluorophore compound containing a phosphoramidite group can be contacted with a DNA or RNA molecule. After a sufficient time for formation of a covalent bond, the fluorophore labeled nucleic acid can be purified from unreacted material. The fluorophore compound may contain several such reactive phosphoramidite groups, resulting in attachment to several nucleic acids.

### Example 64

### Detection of Local Environmental Properties

A fluorophore compound can be used to report on local changes within the biomolecular system in a variety of ways. (a) When the fluorophore has a large ground state dipole moment, local electric fields in the biomolecule or applied to the sample can turn or rotate the fluorophore. The fluorophores further have a large polarizability anisotropy, which means that the polarizability parallel and perpendicular to the molecular long axis are different. Therefore, by pumping with polarized light and/or detecting the polarization of the emitted photons, the orientation of the fluorophore and hence the biomolecule can be determined. (b) The fluorophore compounds of this invention have shown a dependence of emission quantum yield on the rigidity of the local environment (higher quantum yield in a polymer than in a toluene solvent). This means for example that the brightness of the fluorophore can be used to determine the rigidity of the local environment of the fluorophore in the biomolecule. If the biomolecule changes conformation or rigidity, this could be sensed in the emission from the fluorophore. (c) The fluorescence emission lifetime is known to be a sensitive reporter of local quenching effects. For example, some aromatic amino acids cause the emission lifetime of a nearby fluorophore to be reduced. The lifetime of the emitted photons from the fluorophore can be measured to detect changes in the locations of nearby quenching amino acids.

### Example 65

### Detection of Single Biomolecules In Vitro or In Vivo

The fluorophore compounds of this invention have been shown to enable detection at the single-molecule limit in polymers (Willets, K. A., et al., J. Am. Chem. Soc. Commun., 125: 1174-1175 (2003)). This means that the fluorescence quantum yield is sufficiently high, any bottlenecks are sufficiently weak, and photobleaching occurs only with low probability, so that the emitted photons from a single copy can be reliably detected and imaged. When attached to a biomolecule, a single copy of the biomolecule could then be imaged. This removes the ensemble averaging present in conventional experiments, allowing the presence of heterogeneity from copy to copy to be detected and sensed.

### Example 66

### Detection of Single or Many Biomolecules In Vitro or In Vivo by Second Harmonic Generation

The fluorophores of the present invention are known to possess high hyperpolarizability values. This means that when irradiated with wavelength λ₁, the second harmonic of λ₁ at λ₁/2 can be generated. The detection of the shorter wavelength can be done by any of several microscopic techniques with low backgrounds and higher spatial resolution can result due to the nonlinear dependence of the effect on the pumping intensity. A recent report describes this type of imaging with large numbers of native molecules (Dombeck, D. A. et al., Proc. Natl. Acad Sci. U.S.A. 100: 7081-7086 (2003)); but to see a signal, the molecules had to be arrayed in a fashion to remove inversion symmetry. However, with a high-efficiency fluorophore like those of the present invention, the location of the second harmonic emission could be controlled, and a second harmonic signal may be generated by a single molecule with no requirement on having an ordered array.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the methods described herein without departing from the concept and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the scope and concept of the invention.

1. A composition comprising a fluorophore compound, the fluorophore compound having the chemical structure: 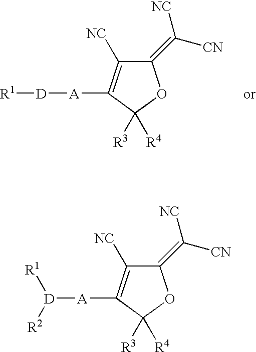
wherein:
D is a donor group having at least one free electron pair conjugated with A;
A is a moiety having at least one multiple bond conjugated with the donor group and the 2-dicyanomethylen-3-cyano-2,5-dihydrofuran group;
R¹ is an alkyl group, alkoxy alkyl group, aromatic group, substituted aromatic group, or hydrogen;
R² is an alkyl group, alkoxy alkyl group, aromatic group, substituted aromatic group, or hydrogen;
R³ is an alkyl group, fluoroalkyl group, aromatic group, or substituted aromatic group;
R⁴ is an alkyl group, fluoroalkyl group, aromatic group, or substituted aromatic group; and the fluorophore compound is not DCDHF-6
(2-[3-Cyano-4-(4-dihexylamino-phenyl)-5,5-dimethyl-5H-furan-2-ylidene]-malononitrile; where A is a benzene ring, D is dihexylamine, R³ is methyl, and R⁴ is methyl).

2. The composition of claim 1, wherein the donor group comprises a nitrogen atom conjugated with A.

3. The composition of claim 1, wherein the donor group comprises an oxygen atom conjugated with A.

4. The composition of claim 1, wherein the donor group comprises a sulfur atom conjugated with A.

5. The composition of claim 1, wherein the donor group comprises a phosphorous atom conjugated with A.

6. The composition of claim 1, wherein A is aromatic.

7. The composition of claim 1, wherein A is benzene, styrene, naphthalene, anthracene, phenanthrene, or pyrene.

8. The composition of claim 1, wherein A is thiophene, furan, pyrrole, imidazole, pyrazole, oxazole, thiazole, diazole, oxadiazole, or thiadiazole.

9. The composition of claim 1, wherein A comprises a vinyl carbon-carbon double bond or an Imine carbon-nitrogen double bond.

10. The composition of claim 1, wherein A comprises a tolane group.

11. The composition of claim 1, wherein the alkyl group is methyl, ethyl, propyl, butyl, pentyl, hexyl, heptyl, C₁₀H₂₁, C₁₂H₂₅, C₁₄H₂₉, C₁₆H₃₃, C₁₈H₃₇, C₂₀H₄₁, or C₂₂H₄₅.

12. The composition of claim 1, wherein the alkoxy alkyl group is methoxymethyl, methoxyethyl, ethoxymethyl, or ethoxyethyl.

13. The composition of claim 1, wherein the fluoroalkyl group is trifluoromethyl or pentafluoroethyl.

14. The composition of claim 1, wherein the compound further comprises at least one functional group suitable for formation of a covalent bond with a biomolecule or biological structure.

15. The composition of claim 14, wherein the functional group is a thiol group, a maleimide group, an iodoacetamide group, an N-hydroxy-succinimide group, a phosphoramidite group, or a methanethiosulfonate group.

16. The composition of claim 14, wherein D comprises the functional group, R¹ comprises the functional group, R² comprises the functional group, R³ comprises the functional group, R⁴ comprises the functional group, or A comprises the functional group.

17. The composition of claim 1, wherein the compound is DCDHF-MOE
(2-(4-{4-[Bis-(2-methoxy-ethyl)-amino]-phenyl}-3-cyano-5,5-dimethyl-5H-furan-2-ylidene)-malononitrile), DCDHF-1
(2-[3-Cyano-4-(4-dimethylamino-phenyl)-5,5-dimethyl-5H-furan-2-ylidene]-malononitrile), DCDHF-C6M
(2-[4-(4-Azepan-1-yl-phenyl)-3-cyano-5,5-dimethyl-5H-furan-2-ylidene]-malononitrile), DCDHF-C5MDM
(2-{3-Cyano-4-[4-(3,5-dimethyl-piperidin-1-yl)-phenyl]-5,5-dimethyl-5H-furan-2-ylidene}-malononitrile), DCDHF-2
(2-[3-Cyano-4-(4-diethylamino-phenyl)-5,5-dimethyl-5H-furan-2-ylidene]-malononitrile), DCDHF-3
(2-[3-Cyano-4-(4-dipropylamino-phenyl)-5,5-dimethyl-5H-furan-2-ylidene]-malononitrile), DCDHF-4
(2-[3-Cyano-4-(4-dibutylamino-phenyl)-5,5-dimethyl-5H-furan-2-ylidene]-malononitrile), DCDHF-5
(2-[3-Cyano-4-(4-dipentylamino-phenyl)-5,5-dimethyl-5H-furan-2-ylidene]-malononitrile), DCDHF-8
(2-[3-Cyano-4-(4-dioctylamino-phenyl)-5,5-dimethyl-5H-furan-2-ylidene]-malononitrile), DCDHF-2EH
(2-(4-{4-[Bis-(2-ethyl-hexyl)-amino]-phenyl}-3-cyano-5,5-dimethyl-5H-furan-2-ylidene)-malononitrile), DCDHF-6-C7M
(2-[3-Cyano-4-(4-dihexylamino-phenyl)-1-oxa-spiro[4.7]dodec-3-en-2-ylidene]-malononitrile), DCDHF-6-DB
(2-[5,5-Dibutyl-3-cyano-4-(4-dihexylamino-phenyl)-5H-furan-2-ylidene]-malononitrile), DCDHF-C6M-CF3
(2-[4-(4-Azepan-1-yl-phenyl)-3-cyano-5-methyl-5-trifluoromethyl-5H-furan-2-ylidene]-malononitrile), DCDHF-6-CF3
(2-[3-Cyano-4-(4-dihexylamino-phenyl)-5-methyl-5-trifluoromethyl-5H-furan-2-ylidene]-malononitrile), DCDHF-2-CF3
(2-[3-Cyano-4-(4-diethylamino-phenyl)-5-methyl-5-trifluoromethyl-5H-furan-2-ylidene]-malononitrile), TH-DCDHF-6
(2-[3-Cyano-4-(5-dihexylamino-thiophen-2-yl)-5,5-dimethyl-5H-furan-2-ylidene]-malononitrile), TH-DCDHF-C6M
(2-[4-(5-Azepan-1-yl-thiophen-2-yl)-3-cyano-5,5-dimethyl-5H-furan-2-ylidene]-malononitrile), TH-DCDHF-6-V
(2-{3-Cyano-4-[2-(5-dihexylamino-thiophen-2-yl)-vinyl]-5,5-dimethyl-5H-furan-2-ylidene}-malononitrile), DCDHF-2-V
(2-{3-Cyano-4-[2-(4-diethylamino-phenyl)-vinyl]-5,5-dimethyl-5H-furan-2-ylidene}-malononitrile), DCDHF-J-V
(2-{3-Cyano-5,5-dimethyl-4-[2-(2,3,6,7-tetrahydro-1H,5H-pyrido[3,2,1-ij]quinolin-9-yl)-vinyl]-5H-furan-2-ylidene}-malononitrile), DCDHF-6-V
(2-{3-Cyano-4-[2-(4-dihexylamino-phenyl)-vinyl]-5,5-dimethyl-5H-furan-2-ylidene}-malononitrile), DCDHF-2EH-V
(2-[4-(2-{4-[Bis-(2-ethyl-hexyl)-amino]-phenyl}-vinyl)-3-cyano-5,5-dimethyl-5H-furan-2-ylidene]-malononitrile), DCDHF-MOE-V
(2-[4-(2-{4-[Bis-(2-methoxy-ethyl)-amino]-phenyl}-vinyl)-3-cyano-5,5-dimethyl-5H-furan-2-ylidene]-malononitrile), DCDHF-DPH-V
(2-{3-Cyano-4-[2-(4-diphenylamino-phenyl)-vinyl]-5,5-dimethyl-5H-furan-2-ylidene}-malononitrile), DCTA-6C-DCDHF-V
(2-[4-(2-{4-[(6-{4-[Bis-(4-carbazol-9-yl-phenyl)-amino]-phenoxy}-hexyl)-ethyl-amino]-phenyl}-vinyl)-3-cyano-5,5-dimethyl-5H-furan-2-ylidene]-malononitrile), PFP-DDCDHF
(2-{3-Cyano-5,5-dimethyl-4-[1-(4-tridecafluorohexyl-phenyl)-1H-pyridin-4-ylidenemethyl]-5H-furan-2-ylidene}-malononitrile), HP-DDCDHF
(2-{3-Cyano-4-[1-(4-hexyl-phenyl)-1H-pyridin-4-ylidenemethyl]-5,5-dimethyl-5H-furan-2-ylidene}-malononitrile), DOCP-DDCDHF
(4-[4-(4-Cyano-5-dicyanomethylene-2,2-dimethyl-2,5-dihydrofuran-3-ylmethylene)-4H-pyridin-1-yl]-benzoic acid dodecyl ester), P-DDCDHF
(2-[3-Cyano-4-(2,6-dimethyl-1-phenyl-1H-pyridin-4-ylidenemethyl)-5,5-dimethyl-5H-furan-2-ylidene]-malononitrile), 2EHO-DDCDHF
(2-(3-Cyano-4-{1-[4-(2-ethyl-hexyloxy)-phenyl]-2,6-dimethyl-1H-pyridin-4-ylidenemethyl}-5,5-dimethyl-5H-furan-2-ylidene)-malononitrile), M2EHO-DDCDHF
(2-(3-Cyano-4-{1-[3-(2-ethyl-hexyloxy)-phenyl]-2,6-dimethyl-1H-pyridin-4-ylidenemethyl}-5,5-dimethyl-5H-furan-2-ylidene)-malononitrile), or DCDHF-2-2V
(2-{3-Cyano-4-[4-(4-diethylamino-phenyl)-buta-1,3-dienyl]-5,5-dimethyl-5H-furan-2-ylidene}-malononitrile).

18. A method of preparing a fluorescently labeled biomolecule, the method comprising contacting a biomolecule and a fluorophore compound under conditions suitable for bonding of the fluorophore compound with the biomolecule; wherein the fluorophore compound has the chemical structure: 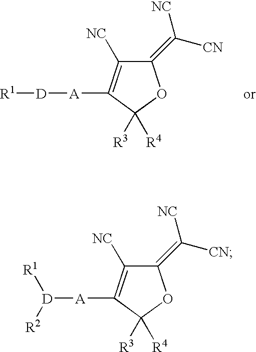
wherein:
D is a donor group having at least one free electron pair conjugated with A;
A is a moiety having at least one multiple bond conjugated with the donor group and the 2-dicyanomethylen-3-cyano-2,5-dihydrofuran group;
R¹ is an alkyl group, alkoxy alkyl group, aromatic group, substituted aromatic group, or hydrogen;
R² is an alkyl group, alkoxy alkyl group, aromatic group, substituted aromatic group, or hydrogen;
R³ is an alkyl group, fluoroalkyl group, aromatic group, or substituted aromatic group; and
R⁴ is an alkyl group, fluoroalkyl group, aromatic group, or substituted aromatic group.

19. The method of claim 18, wherein the biomolecule is a nucleic acid.

20. The method of claim 18, wherein the biomolecule is a protein.

21. The method of claim 18, wherein the biomolecule is a peptide.

22. The method of claim 18, wherein the biomolecule is a monosaccharide or a polysaccharide.

23. The method of claim 18, wherein the biomolecule is a nucleotide.

24. The method of claim 18, wherein the biomolecule is a lipid.

25. The method of claim 18, wherein the bonding comprises formation of a covalent bond.

26. The method of claim 18, wherein the bonding comprises formation of an ionic bond, a pi-pi stacking interaction, a hydrophobic interaction, or van der Waals interaction.

27. The method of claim 18, wherein the compound further comprises at least one functional group suitable for formation of a covalent bond with a biomolecule or biological structure.

28. The method of claim 27, wherein the functional group is a thiol group, a maleimide group, an iodoacetamide group, an N-hydroxy-succinimide group, a phosphoramidite group, or a methanethiosulfonate group.

29. The method of claim 27, wherein D comprises the functional group, R¹ comprises the functional group, R² comprises the functional group, R³ comprises the functional group, R⁴ comprises the functional group, or A comprises the functional group.

30. The method of claim 18, further comprising a step of detecting the biomolecule after the contacting step.

31. The method of claim 18, further comprising a step of analyzing the fluorescently labeled biomolecule, the analyzing step selected from the group consisting of detecting fluorescence, detecting polarization, detecting anisotropy, detecting fluorescence lifetime, detecting spectrum, determining correlations, and detecting second harmonic.

32. A method of preparing a fluorescently labeled biological structure within a cell, the method comprising:
providing a cell or cells comprising a biological structure; and
contacting the cell or cells with a fluorophore compound under conditions suitable for cellular uptake of the fluorophore compound and bonding of the fluorophore compound with the biological structure; wherein the fluorophore compound has the chemical structure: 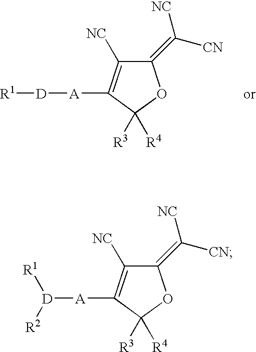
wherein:
D is a donor group having at least one free electron pair conjugated with A;
A is a moiety having at least one multiple bond conjugated with the donor group and the 2-dicyanomethylen-3-cyano-2,5-dihydrofuran group;
R¹ is an alkyl group, alkoxy alkyl group, aromatic group, substituted aromatic group, or hydrogen;
R² is an alkyl group, alkoxy alkyl group, aromatic group, substituted aromatic group, or hydrogen;
R³ is an alkyl group, fluoroalkyl group, aromatic group, or substituted aromatic group; and
R⁴ is an alkyl group, fluoroalkyl group, aromatic group, or substituted aromatic group.

33. The method of claim 32, wherein the biological structure is a lipid bilayer, a membrane, a micelle, the cytoskeleton, a nucleosome, a ribosome, a peroxisome, a liposome, a plastid, a transmembrane protein, a chloroplast, or a mitochondrion.

34. The method of claim 32, wherein the bonding comprises formation of a covalent bond.

35. The method of claim 32, wherein the bonding comprises formation of an ionic bond, a pi-pi stacking interaction, a hydrophobic interaction, or van der Waals interaction.

36. The method of claim 32, wherein the compound further comprises at least one functional group suitable for formation of a covalent bond with a biomolecule or biological structure.

37. The method of claim 32, wherein the functional group is a thiol group, a maleimide group, an iodoacetamide group, an N-hydroxy-succinimide group, a phosphoramidite group, or a methanethiosulfonate group.

38. The method of claim 32, wherein D comprises the functional group, R¹ comprises the functional group, R² comprises the functional group, R³ comprises the functional group, R⁴ comprises the functional group, or A comprises the functional group.

39. The method of claim 32, further comprising a step of detecting the biological structure after the contacting step.

40. The method of claim 32, further comprising a step of analyzing the fluorescently labeled biological structure, the analyzing step selected from the group consisting of detecting fluorescence, detecting polarization, detecting anisotropy, detecting fluorescence lifetime, detecting spectrum, determining correlations, and detecting second harmonic.
